(19) United States
(12) Patent Application Publication
(10) Pub. No.: US 2009/0088982 A1
(43) Pub. Date: Apr. 2, 2009

(54) Co-detection of single polypeptide and polynucleotide molecules

(76) Inventors: Noelle H. Fukushima, Tucson, AZ (US); Robert S. Puskas, Manchester, MO (US)

Correspondence Address: WILSON SONSINI GOODRICH & ROSATI, 650 PAGE MILL ROAD, PALO ALTO, CA, 94304-1050, US

(21) Appl. No.: 11/659,057

(22) PCT Filed: Jul. 31, 2004
(86) PCT No.: PCT/US04/24690

§ 371 Date: Dec. 17, 2008

Related U.S. Application Data
(60) Provisional application No. 60/492,137, filed on Jul. 31, 2003.

(51) Int. Cl. G01N 33/483; G06F 19/00

(52) U.S. Cl. 702/23; 356/246

(57) ABSTRACT

The invention relates to a method for co-detecting and distinguishing individual polypeptide and polynucleotide molecules in a single sample analysis. The polypeptide and polynucleotide targets which emit electromagnetic radiation are labeled with probes that emit electromagnetic radiation at the same emission wavelength, are suspended in fluid and moved through an interrogation volume allowing co-detection of low concentrations of individual molecules and discrimination based on characteristics other than emission wavelength.

### CROSS-REFERENCE TO RELATED APPLICATIONS

This application claims priority from U.S. Provisional Application Ser. No. 60/492,137 filed on Jul. 31, 2003, which is incorporated herein by reference in its entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

The invention relates to co-detecting single polypeptide and polynucleotide molecules in a single sample analysis.

2. Description of Related Art

The ability to co-detect proteins and nucleic acids is important for determining reactions and interactions during complex processes such as those that occur in biological systems. Most organisms contain both of these biomolecules, and the biological processes often involve measurable changes in both proteins and nucleic acids that help to elucidate their function. Known in vitro methods for co-detection of polypeptide and polynucleotide molecules are limited in that they require large amounts of sample, complex and expensive equipment, or a significant amount of time to perform. Many of these known methods measure large groups or ensembles of biomolecules, which contain perhaps hundreds or thousands of individual molecules within a single sample. These measurements obscure valuable information about properties of individual molecules. For example, if two properties are measured, ensemble measurement cannot determine whether both properties occur on one molecule or whether the first property and the second property each occur on only one half of the molecules. The current invention provides a method for co-detection of single polypeptide and polynucleotide molecules at very low concentrations that is fast, simple and inexpensive.

Numerous techniques have been developed for co-detection of polypeptides and polynucleotides using ensemble measurement of biomolecules. Co-detection can be performed using gel electrophoresis, flow cytometry, micro array technology, immunohistochemistry, in-situ hybridization and single molecule detection (“SMD”).

The gel electrophoresis technique often detects one of the molecules indirectly. For example, a nucleic acid binding protein can be detected indirectly by a shift in electrophoretic velocity when the protein is bound to the nucleic acid. In these assays, the nucleic acid is detected by staining while the presence of the protein is inferred by a shift in electrophoretic velocity. Similar mobility shift assays have also been performed on single molecules with varying degrees of accuracy (LeCaptain, 2001). Other gel electrophoresis techniques distinguish the ensemble proteins and nucleic acids based on differences in emission characteristics of the molecules, either directly or after applying labels that emit electromagnetic radiation at different wavelengths. Jing et al., used SYBR Green and SYPRO Ruby labels, and Alba, et al. used SYPRO Red and YOYO labels (Molecular Probes, Eugene Oreg.) (Jing, 2003; Alba, 2001-2). One of the major disadvantages of these techniques is the need for large (pg to ng) quantities and high concentrations of target molecules (Haugland, 2002). Also, the run and analysis takes a long time (from a few hours to overnight). Further, quantitation requires densitometry measurements that are generally not precise, making it difficult to obtain accurate information.

Flow cytometry technology requires particles that scatter light using either cells or beads, and identifies fluorescent targets bound to the particles. The targets are suspended in fluid and flow past laser illumination sources and photo multiplier tube detectors. Proteins and nucleic acids are labeled with distinct fluorescent tags and the molecules are distinguished based on the different emission wavelengths of the labels. Flow cytometry methods that measure both proteins and nucleic acids include cellular assays for apoptosis (Waters, 2002; Hasper, 2000), cell cycle (Crissman, 1990; Niculescu, 1998), and cell replication (Faretta, 1998; Holm, 1998). In addition, molecular interactions between proteins and nucleic acids have been measured using beads detected by flow cytometry (Brodsky, 2002). Assays using beads to capture and detect molecules are sensitive in the high femtogram range. (Kellar, 2002; Dunbar, 2003). Although these techniques are fast to perform and a large number of events can be analyzed, detectable fluorescence intensity from single molecules cannot be achieved because it requires multiple bound target molecules and only single cells or particles are detected. Another disadvantage of this technology is that it does not allow for measurement of isolated molecules but requires they be attached to cells or beads. Precise quantitation is also difficult to accomplish.

Micro array technology is another method used for ensemble measurement of polypeptides and polynucleotides, and a co-detection assay has been developed (Perrin, 2003). In this co-detection experiment, antigens and oligonucleotides bound to a plate are used to capture antibodies and nucleic acids respectively from clinical samples using a micro titer plate format and colorimetric readout. Target proteins and nucleic acids are distinguished based on their locations on the array. The assay is configured as a diagnostic tool for virus infection and requires samples with molecule concentrations in the range 18-570 pM depending on the specific target (Perrin, 2003). However, this technique's usefulness is limited due to its low sensitivity. In addition, the equipment is very expensive and a significant amount of time is required (from hours to days) to build the arrays and to run and analyze samples.

Immunohistochemistry and in-situ hybridization can be used for co-detection of polypeptide and polynucleotide molecules in or on cells or tissues. Again, proteins and nucleic acids are generally distinguished based on different emission wavelengths from their respective labels. The preferred method uses fluorescently labeled tags to identify and locate specific polypeptides or polynucleotides. Probes can be labeled antibodies, oligonucleotides, or reactive metals, intercalating dyes, and fluorescent beads (Haugland, 2002). In these assays, cells or tissues are fixed on a surface (usually a glass slide or plastic dish), and illuminated with a UV light source. Microscopic imaging is used for the detection (Nagaso, 2001; Fischer, 2002). Co-detection using other distinguishing non-fluorescent labels such as radioactive and colorimetric labels has also been described (Chiu, 1996). However, again, detectable fluorescence intensity cannot be achieved with single molecules because this technique requires multiple molecules and only individual cell substructures are detected. Other disadvantages include relatively slow performance time (days), analysis of generally low numbers of cells or images and the requirement of expensive and fragile equipment. Also, a high level of skill and expertise is required to perform the techniques and additional sophisticated software is required for quantitation and analysis.

In the past 10 years, single molecule detection (SMD) has become a viable approach to the sensitive detection of biomolecules (Ambrose, 1999; Keller, 2002; Schwille, 2002; Wrotnowski, 2002). With SMD, individual molecules can be detected, quantitated, and discriminated in a mixture of molecules. Discrimination commonly uses measurement of electromagnetic characteristics, electrophoretic velocity and target-specific labels, such as oligomeric labels for nucleic acids and antibodies for proteins. SMD typically is used to identify or assay a single molecular species. Ma et al. have recently described co-detection of a protein and nucleic acid; however, the method described is very limited in that it requires labeling each of the analytes with a fluorescent dye that has emission wavelengths different from that of the other analyte (Ma, 2000). A CCD camera imaging system is used to identify complexes of protein bound to DNA, as well as individual labeled protein and DNA molecules in a mixture. Discrimination is based on the different emission wavelengths of the labeled molecules using a transmission grating. There are many disadvantages with these methods in that they require very specific materials. Sets of dyes that are compatible with both the analytes and the detection system are necessary. Further, the imaging system is very complex and there is a lack of automated data analysis.

Therefore, what is needed is a simple method to quickly and accurately measure individual molecules that would require smaller quantities of molecules and no attachment to a particle.

### BRIEF SUMMARY OF THE INVENTION

Accordingly, it is an object of the invention to overcome these and other problems associated with the related art. These and other objects, features and technical advantages are achieved by providing methods of detecting single molecules by interaction with labels.

This invention provides a method for co-detecting a polynucleotide molecule and a polypeptide molecule contained within one sample comprising, in any order: (a) moving or disposing said polynucleotide molecule and said polypeptide molecule through at least one interrogation volume; and (b) measuring at least one electromagnetic characteristic of said polynucleotide molecule and measuring at least one electromagnetic characteristic of said polypeptide molecule, wherein said polynucleotide molecule and said polypeptide molecule are co-detected. In one aspect, the polynucleotide molecule and the polypeptide molecule are discriminated. In another aspect, the electromagnetic characteristic of the polynucleotide molecule and polypeptide molecule are measured simultaneously or sequentially.

In yet another aspect, a plurality of the same polynucleotide molecule and a plurality of the same polypeptide molecule are co-detected. In one alternative, a plurality of different polynucleotide molecules and a plurality of different polypeptide molecules are co-detected. In another alternative, a plurality of the same or different polynucleotide molecules and a plurality of the same or different polypeptide molecules are co-detected. Preferably, the polynucleotide is selected from the group consisting of a single-stranded DNA, a double-stranded DNA, an oligonucleotide, an RNA, a dendrimer, a nucleic acid hybrid and any combination thereof. Preferably, the polypeptide is selected from the group consisting of an oligopeptide and a protein.

In accordance with a further aspect of the invention, the polypeptide electromagnetic characteristic and the polynucleotide electromagnetic characteristic are measured within at least one interrogation volume, which may be optionally fluidly connected to one or more other interrogation volumes, said interrogation volume being in electromagnetic communication with at least one detector and at least one excitation source.

In another aspect, the sample comprises a plurality of the same or different polynucleotide molecules and a plurality of the same or different polypeptide molecules, and act (b) further comprises: measuring at least one of a first or a second electromagnetic characteristic of at least one polynucleotide molecule as the at least one polynucleotide molecule interacts with an excitation source within a first interrogation volume; measuring at least one of a first or a second electromagnetic characteristic of the at least one polynucleotide molecule as the at least one polynucleotide molecule interacts with an excitation source within a second interrogation volume; comparing the measured electromagnetic characteristics measured within the first and the second interrogation volumes of the at least one polynucleotide; measuring at least one of a first or a second electromagnetic characteristic of at least one polypeptide molecule as the at least one polypeptide molecule interacts with an excitation source within the first, the second or a third interrogation volume; measuring at least one of a first or a second electromagnetic characteristic of the at least one polypeptide molecule as the at least one polypeptide molecule interacts with an excitation source within the first, the second or a fourth interrogation volume; and comparing the measured electromagnetic characteristics measured within the first, the second, the third and the fourth interrogation volumes of the at least one polypeptide.

In another aspect, the act of comparing comprises distinguishing by statistical analysis the measured electromagnetic characteristics from the at least one polypeptide molecule and the at least one polypeptide molecule from background electromagnetic characteristics. In one alternative, the act of comparing comprises cross-correlating the measured electromagnetic emissions determined from the at least one polypeptide molecule and the at least one polypeptide molecule to determine the velocity of the molecules. In another alternative, the act of comparing comprises cross-correlating the measured electromagnetic emissions to determine the velocity of the molecules.

In another aspect, act (a) comprises subjecting the sample to a motive force selected from the group consisting of electro-kinetic, pressure, vacuum, surface tension, gravity, centrifugal, and any combination thereof. In yet another aspect, the act of moving the molecules between a first interrogation volume and a second interrogation volume further comprises subjecting the molecules to a separation method selected from the group consisting of capillary gel electrophoresis, micellar electro-kinetic chromatography, isotachophoresis, and any combination thereof.

In accordance with a further aspect of the invention, at least one of the measurable characteristics of the polynucleotide molecule and the measurable characteristics of the polypeptide molecule is produced by one of an intrinsic parameter of the molecule and an extrinsic parameter of the molecule. Preferably, the extrinsic parameter is provided by marking the polynucleotide molecule and the polypeptide molecule with at least one label to provide the extrinsic parameter. In one alternative, the polynucleotide molecule and polypeptide molecule are labeled prior to performing act (a). In another alternative, at (east one polynucleotide and at least one polypeptide are labeled in separate reactions and are combined to create the sample prior to performing act (a). In yet another alternative, at least one polynucleotide and at least one polypeptide are labeled in the same reaction prior to performing act (a).

In accordance with a further aspect of the invention, the label affects the mobility of at least one of the polynucleotide molecule and the polypeptide molecule and is selected from the group consisting of a charge tag, a mass tag, a charge/mass tag, and any combination thereof. In one alternative, the method further comprises moving the polynucleotide molecule in a direction generally opposite to a direction of the polypeptide molecule. In another alternative, the sample comprises a plurality of different polynucleotide molecules, and further comprises moving a first polynucleotide molecule in a direction generally opposite to a direction of a second polynucleotide molecule. In another alternative, the sample comprises a plurality of different polypeptide molecules, further comprising moving a first polypeptide molecule in a direction generally opposite to a direction of a second polypeptide molecule.

In another aspect, the method further comprises moving the polynucleotide molecule in a direction generally perpendicular to a direction of the polypeptide molecule. In one alternative, the sample comprises a plurality of different polynucleotide molecules, further comprising moving a first polynucleotide molecule in a direction generally perpendicular to a direction of a second polynucleotide molecule. In another alternative, the sample comprises a plurality of different polypeptide molecules, further comprising moving a first polypeptide molecule in a direction generally perpendicular to a direction of a second polypeptide molecule.

In accordance with a further aspect of the invention, the label emits electromagnetic radiation selected from the group consisting of fluorescence, chemiluminescence, elastic light scattering, inelastic light scattering, and any combination thereof. In another aspect, the measured electromagnetic characteristic is selected from the group consisting of emission wavelength, emission intensity, burst size, burst duration, fluorescence polarity, fluorescence lifetime and any combination thereof.

In accordance with a further aspect of the invention, the polynucleotide label and the polypeptide label comprise indistinguishable emission wavelengths. Preferably, the polynucleotide label or polypeptide label further comprises a characteristic other than emission wavelength which is distinguishable. Preferably, the characteristic other than emission wavelength is selected from the group consisting of mobility, emission intensity, burst size, burst duration, fluorescence polarity, fluorescence lifetime, and any combination thereof. More preferably, the electromagnetic characteristics of the polynucleotide label and the polypeptide label are indistinguishable and wherein the polynucleotide and the polypeptide are distinguishable by their mobility. Still more preferably, the electromagnetic characteristics of the polynucleotide label and the polypeptide label are indistinguishable and wherein a predetermined range of labels is attached to each polynucleotide molecule and polypeptide molecule, and further wherein the polynucleotide molecule and polypeptide molecule are distinguishable by a characteristic signal produced by the difference in the range of labels attached to each molecule.

In accordance with a further aspect of the invention, the polynucleotide molecule and polypeptide molecule are labeled directly by means of specific or nonspecific interactions selected from a group consisting of covalent binding, ionic binding, hydrophobic binding, affinity binding, and any combination thereof. In one alternative, the polynucleotide and polypeptide are labeled indirectly by means of incubating with at least one binding partner to form a specific complex and wherein at least one binding partner comprises at least one label. Preferably, the binding partners are selected from the group consisting of polynucleotide/polynucleotide interactions, polynucleotide/polypeptide interactions and polypeptide/polypeptide interactions, and any combination thereof. More preferably, the interaction between binding partners is mediated through intermolecular forces selected from the group consisting of hydrophobic interactions, hydrogen bonding, ionic bonding, van der Waals attraction, covalent bonding, and any combination thereof.

In accordance with a further aspect of the invention, at least one polynucleotide molecule is detected and at least one polypeptide molecule is detected, and the method further comprises counting the molecules. Preferably, said counting comprises determining a concentration of polynucleotide molecules within the sample and a concentration of polypeptide molecules within the sample, further comprising comparing the counted polynucleotide molecules with a polynucleotide standard of a known concentration; and comparing the counted polypeptide molecules with a polypeptide standard of a known concentration. In one alternative, said counting comprises determining a concentration of polynucleotide molecules within the sample and a concentration of polypeptide molecules within the sample, further comprising comparing the counted polynucleotide molecules with an internal polynucleotide standard of a known concentration; and comparing the measured molecules of the polypeptide with an internal polypeptide standard of a known concentration. In yet another alternative, said counting comprises determining a concentration of polynucleotide molecules and polypeptide molecules without use of an external standard or an internal standard.

This invention also provides a method for co-detecting a polynucleotide molecule and a polypeptide molecule within one sample comprising: (a) labeling at least one polynucleotide molecule and at least one polypeptide molecule with at least one directly or indirectly detectable label in a mixture of polynucleotide molecules, polypeptide molecules and excess labels; (b) separating or rendering undetectable, or otherwise distinguishing unbound labels from the labeled polynucleotide molecules and labeled polypeptide molecules; (c) interacting the labeled polynucleotide molecules and labeled polypeptide molecules with an agent causing the release of directly or indirectly bound labels; and (d) detecting at least one polynucleotide molecule released label and at least one polypeptide molecule released label, thereby rendering the polynucleotide molecule and a polypeptide molecule co-detectable.

In accordance with a further aspect of the invention, a method is provided wherein act (d) comprises: moving or disposing the at least one polynucleotide molecule released label and the at least one polypeptide molecule released label through at least one interrogation volume; and measuring at least one electromagnetic characteristic of the at least one polynucleotide molecule released label and measuring at least one electromagnetic characteristic of the at least one polypeptide molecule released label. In one aspect, the polynucleotide molecule released label and the polypeptide molecule released label are discriminated.

In another aspect, the electromagnetic characteristic of the polynucleotide molecule released label and polypeptide molecule released label are measured simultaneously or sequentially. In yet another aspect, a plurality of the same polynucleotide molecule and a plurality of the same polypeptide molecule are indirectly co-detected. In one alternative, a plurality of different polynucleotide molecules and a plurality of different polypeptide molecules are indirectly co-detected. In another alternative, a plurality of the same or different polynucleotide molecules and a plurality of the same or different polypeptide molecules are indirectly co-detected.

In accordance with a further aspect of the invention, the polypeptide molecule released label electromagnetic characteristic and the polypeptide molecule released label electromagnetic characteristic are measured within at least one fluidly connected interrogation volume, said interrogation volume being in electromagnetic communication with at least one detector and at least one excitation source.

In another aspect, the sample comprises a plurality of the same or different polynucleotide molecule released labels and a plurality of the same or different polypeptide molecule released labels, and wherein the act of measuring further comprises: measuring at least one of a first or a second electromagnetic characteristic of at least one polynucleotide molecule released label as the at least one polynucleotide molecule released label interacts with an excitation source within a first interrogation volume; measuring at least one of a first or a second electromagnetic characteristic of the at least one polynucleotide molecule released label as the at least one polynucleotide molecule released label interacts with an excitation source within a second interrogation volume; comparing the measured electromagnetic characteristics measured within the first and the second interrogation volumes of the at least one polynucleotide molecule released label; measuring at least one of a first or a second electromagnetic characteristic of at least one polypeptide molecule released label as the at least one polypeptide molecule released label interacts with an excitation source within the first, the second or a third interrogation volume; measuring at least one of a first or a second electromagnetic characteristic of the at least one polypeptide molecule released label as the at least one polypeptide molecule released label interacts with an excitation source within the first, the second or a fourth interrogation volume; and comparing the measured electromagnetic characteristics measured within the first, the second, the third and the fourth interrogation volumes of the at least one polypeptide molecule released label.

In another aspect, the act of comparing comprises distinguishing by statistical analysis the measured electromagnetic characteristics of the at least one polynucleotide molecule released label and the at least one polypeptide molecule released label from background electromagnetic characteristics. In one alternative, the act of comparing comprises cross-correlating the measured electromagnetic emissions determined to be of the at least one polynucleotide molecule released label and the at least one polypeptide molecule released label to determine the velocity of the labels. In another alternative, the act of comparing comprises cross-correlating the measured electromagnetic emissions to determine the velocity of the labels.

In yet another aspect, the act of moving comprises subjecting the sample to a motive force selected from the group consisting of electro-kinetic, pressure, vacuum, surface tension, gravity, centrifugal, and any combination thereof. In another aspect, the act of moving the released labels between a first interrogation volume and a second interrogation volume further comprises subjecting the released labels to a separation method selected from the group consisting of capillary gel electrophoresis, micellar electro-kinetic chromatography, isotachophoresis, and any combination thereof.

In accordance with a further aspect of the invention, at least one measurable characteristic of the polynucleotide molecule released label and at least one measurable characteristic of the polypeptide molecule released label is produced by one of an intrinsic parameter of the released labels and an extrinsic parameter of the released labels. Preferably, the extrinsic parameter is provided by attaching a secondary label to the released label to provide the extrinsic parameter. In one alternative, the at least one polynucleotide molecule released label and the at least one polypeptide molecule released label are marked with the secondary label prior to performing the act of moving. In another alternative, the secondary label is attached to the polynucleotide molecule released label and the polypeptide molecule released label in separate reactions and are combined to create the sample prior to performing the act of moving. In yet another alternative, the secondary label is attached to the polynucleotide molecule released label and the polypeptide molecule released label in the same reaction prior to performing the act of moving.

In another aspect, the secondary label affects the mobility of the at least one polynucleotide molecule released label and the at least one polypeptide molecule released label, and wherein the secondary label is selected from the group consisting of a charge tag, a mass tag, a charge/mass tag, and any combination thereof. In one alternative, the method further comprises moving the marked polynucleotide molecule released label in a direction generally opposite to a direction of the marked polypeptide molecule released label. In another alternative, the sample comprises a plurality of different polynucleotide molecule released labels, further comprising moving a first marked polynucleotide molecule released label in a direction generally opposite to a direction of a second marked polynucleotide molecule released label. In another alternative, the sample comprises a plurality of different polypeptide molecule released labels, further comprising moving a first marked polypeptide molecule released label in a direction generally opposite to a direction of a second marked polypeptide molecule released label.

In accordance with a further aspect of the invention, the method further comprises moving the marked polynucleotide molecule released label in a direction generally, at an angle, preferably perpendicular to a direction of the marked polypeptide molecule released label. In one alternative, the sample comprises a plurality of different polynucleotide molecule released labels, further comprising moving a first marked polynucleotide molecule released label in a direction generally perpendicular to a direction of a second marked polynucleotide molecule released label. In another alternative, the sample comprises a plurality of different polypeptide molecule released labels, further comprising moving a first marked polypeptide molecule released label in a direction generally perpendicular to a direction of a second marked polypeptide molecule released label.

In accordance with a further aspect of the invention, the polynucleotide molecule released label, polypeptide molecule released label or secondary label emits electromagnetic radiation selected from the group consisting of fluorescence, chemiluminescence, light scattering, and any combination thereof. In another aspect, the measured electromagnetic characteristic is selected from the group consisting of emission wavelength, emission intensity, burst size, burst duration, fluorescence polarity, fluorescence lifetime and any combination thereof.

In accordance with a further aspect of the invention, the polynucleotide molecule released label and the polypeptide molecule released label comprise indistinguishable emission wavelengths. Preferably, the secondary label comprises a characteristic other than emission wavelength which is distinguishable. Preferably, the characteristic other than emission wavelength is selected from the group consisting of mobility, emission intensity, burst size, burst duration, fluorescence polarity, fluorescence lifetime and any combination thereof. More preferably, the electromagnetic characteristics of the polynucleotide molecule released label and the polypeptide molecule released label are indistinguishable, and wherein the at least one polynucleotide molecule released label and the at least one polypeptide molecule released label are indirectly distinguishable by the secondary label mobility.

In another aspect, the at least one polynucleotide molecule released label and the at least one polypeptide molecule released label are marked directly with the secondary label by means of specific or nonspecific interactions selected from a group consisting of covalent binding, ionic binding, hydrophobic binding, affinity binding, and any combination thereof. In one alternative, the at least one polynucleotide molecule released label and the at least one polypeptide molecule released label are marked indirectly with the secondary label by means of incubating with at least one binding partner to form a specific complex and wherein at least one binding partner comprises at least one secondary label. Preferably, the binding partners are selected from the group consisting of protein/protein interactions, protein/nucleic acid interactions, nucleic acid/nucleic acid interactions, and any combination thereof. More preferably, the interaction between binding partners is mediated through intermolecular forces selected from the group consisting of hydrophobic interactions, hydrogen bonding, ionic bonding, van der Waals attraction, covalent bonding, and any combination thereof.

In accordance with a further aspect of the invention, at least one released label is detected, and the method further comprises counting the released labels. Preferably, said counting comprises determining a concentration of at least one polynucleotide molecule released label and at least one polypeptide molecule released label within the sample, further comprising: comparing the counted polynucleotide molecule released labels with a polynucleotide molecule label standard of a known concentration; and comparing the counted polypeptide molecule released labels with a polypeptide molecule label standard of a known concentration. In one alternative, said counting comprises determining a concentration of at least one polynucleotide molecule released label and at least one polypeptide molecule released label within the sample, further comprising comparing the counted polynucleotide molecule released labels with an internal polynucleotide molecule label standard of a known concentration; and comparing the counted polypeptide molecule released labels with an internal polypeptide molecule label standard of a known concentration. In another alternative, said counting comprises determining a concentration of at least one polynucleotide molecule released label and at least one polypeptide molecule released label without use of external or internal standards. In yet another alternative, the number of the at least one polynucleotide molecule released label and the at least one polypeptide molecule released label counted are proportional to the concentration of the polynucleotide molecules and polypeptide molecules in the original sample.

In accordance with a further aspect of the invention, a method is provided wherein at least one secondary label is detected, and the method further comprises counting the secondary labels. In one alternative, said counting comprises determining a concentration of at least one secondary label within the sample, further comprising comparing the counted secondary labels released from the polynucleotide molecule with a secondary label standard of a known concentration; and comparing the counted secondary labels released from the polypeptide molecule with a secondary label standard of a known concentration. In another alternative, said counting comprises determining a concentration of at least one secondary label within the sample, further comprising comparing the counted secondary labels released from the polynucleotide molecule with an internal secondary label standard of a known concentration; and comparing the counted secondary labels released from the polypeptide molecule with an internal secondary label standard of a known concentration. In another alternative, said counting comprises determining a concentration of at least one secondary label without use of external or internal standards. In yet another alternative, the number of the at least one secondary labels counted are proportional to the concentration of the polynucleotide molecules and polypeptide molecules in the original sample.

These and other features, aspects and advantages of the present invention will become better understood with reference to the following description, examples and appended claims.

### BRIEF DESCRIPTION OF THE SEVERAL VIEWS OF THE DRAWINGS

FIG. 1. Electrophoretic discrimination of a protein and nucleic acid, both labeled with Alexa Fluor® 647 with linear polyacrylamide buffer. A) Labeled protein alone. B) Labeled nucleic acid alone. C) Two peaks resolved in a mixture of labeled protein and labeled nucleic acid.

FIG. 2. Electrophoretic discrimination of a protein and nucleic acid, both labeled with Alexa Fluor® 647. A) Labeled protein alone. B) Labeled nucleic acid alone. C) Two peaks resolved in a mixture of labeled protein and labeled nucleic acid.

FIG. 3. Discrimination of a protein and a nucleic acid based on fluorescence intensity. A) Fluorescence intensity is plotted as a function of the time offset for detection of fluorescence at the two detectors. Each spot represents measurements taken on a single molecule. A value of 500 photons/msec was used to divide the “bright molecule” window from the “dim molecule” window. PBXL-3 molecules (left) emit at a higher average intensity than the pUC19 molecules (right). B) The concentration of PBXL-3 and pUC19 components in mixtures measured via counting fluorescent molecules is compared to the predicted values.

FIG. 4. Electrophoretic velocity of protein complex (PBXL-3) shifts when bound to nucleic acid. A) PBXL-3 alone migrates as a peak at 368 ms. B) PBXL-3 bound to nucleic acid migrates as a peak at 294 ms. C) PBXL-3 in the presence of, but not bound to nucleic acid migrates at 409 ms.

FIG. 5. Discrimination of labels released from protein target and nucleic acid targets. A) Detection of label released from a protein target. A linear relationship was observed between the net molecules of Alexa Fluor®647 measured and the original TSH concentration. B) & C) Examples of discrimination of released labels based on their electrophoretic mobility or fluorescence intensity.

FIG. 6. Examples of FRET affects on protein (P) or nucleic acid (NA) labels. A) Oligonucleotide molecule can be labeled with a FRET donor (D) and a second oligonucleotide can be marked with a FRET acceptor (A). Protein molecules can be labeled with an antibody marked with the FRET donor only. B) Nucleic acid and protein molecules are labeled with the same FRET donor. Protein molecules can also be labeled with an antibody marked with a FRET acceptor. In both cases, protein molecules are distinguished from nucleic acid molecules by virtue different wavelengths of emitted radiation.

FIG. 7. Discrimination of a protein and nucleic acid based on molecular mobility in a two dimensional separation system. An electrical field applied perpendicular to the axis of the capillary can cause molecules to move sideways within the capillary. The perpendicular electrical field can be combined with flow through the capillary created by pumping or gravitational force (A) or electrophoresis (B).

### DETAILED DESCRIPTION OF THE INVENTION

### Abbreviations and Definitions

To facilitate understanding of the invention, a number of terms and abbreviations as used herein are defined below as follows:

Binding partner(s): As used herein the term “binding partners” refers to macromolecules that combine through molecular recognition to form a complex. Molecular recognition involves topological compatibility or the matching together of interacting surfaces on each partner. The partners can be described as complementary, and furthermore, the contact surface characteristics are complementary to each other. Binding forces can be hydrophobic, hydrophilic, ionic, hydrogen, van der Waals, and/or covalent. Examples of binding partners include: epitope/antibody, oligonucleotide/nucleic acid, and ligand/receptor.

Charge tags: As used herein, the term “charge tag” refers to any entity bearing a charge that when bound to or associated with the target distinguishes the charge tag+target from the target alone based on detection of the mass, charge, or charge to mass ratio. A charge tag can be a label.

Charge/mass tags: As used herein, the term “charge/mass tag” refers to any charge and mass added to the target that serves to distinguishes the charge/mass tag+target from the probe alone based on detection of the mass, charge, or charge to mass ratio. A charge/mass tag can be a label.

Chemiluminescence: As used herein, the term “chemiluminescence” refers to luminescence produced by the direct transformation of chemical energy into light energy. Also called chemoluminescence.

Co-detection: As used herein, the term “co-detection” refers to the detection of polynucleotide molecules and polypeptide molecules within a single sample. Preferably, the co-detection is provided by detecting a polynucleotide and polypeptide simultaneously, but may also include sequential detection so long as any single molecule is detected at any given time. Co-detected molecules may also be discriminated unless detection of the second molecular species is above a known threshold of the first molecular species.

Cross-correlation: As used herein, the term ‘cross-correlation’ involves subjecting two raw data sets gⱼ and hₖ to analysis, whereby data sets from each detector (preferably photon detectors) are subjected to the following formula:

$${Corr ( g , h )}_{j} ≡ {\sum }_{k = 0}^{N - 1} {g}_{j + k} {h}_{k} , \\ for j = - ( N - 1 ) , - ( N - 2 ) , … , - 1 , 0 , 1 , … , N - 1$$

where N is the total number of data points. The data cross-correlations will be large at values of j where the first data set from a detector [preferably photon counts above a background level] (g) resembles the data set (h) from a second detector [preferably above a background level] at some lag time (j) that corresponds to the time for specific molecules to pass from the first detector to the second detector (preferably in a single molecule analytical system).

Dye: As used herein, the term “dye” refers to a substance used to color materials or to enable generation of luminescent or fluorescent light. A dye may absorb light or emit light at specific wavelengths. A dye may be intercalating, noncovalently bound or covalently bound to a target. Dyes themselves may constitute labels that detect minor groove structures, cruciforms, loops or other conformational elements of molecules. Dyes may include BODIPY and ALEXA dyes, Cy[n] dyes, SYBR dyes, ethidium bromide and related dyes, acridine orange, ethidium bromide, SYBR, dimeric cyanine dyes such as TOTO, YOYO, BOBO, TOPRO POPRO, and POPO and their derivatives, bis-benzimide, OliGreen, PicoGreen and related dyes, cyanine dyes, fluorescein, LDS 751, DAPI, AMCA, Cascade Blue, CL-NERF, Dansyl, Dialkylaminocoumarin, 4′,5′-Dichloro-2′,7′-dimethoxyfluorescein, 2′,7′-Dichlorofluorescein, DM-NERF, Eosin, Erythrosin, Fluoroscein, Hydroxycourmarin, Isosulfan blue, Lissamine rhodamine B, Malachite green, Methoxycoumarin, Naphthofluorescein, NBD, Oregon Green, PyMPO, Pyrene, Rhodamine, Rhodol Green, 2′,4′,5′,7′-Tetrabromosulfonefluorescein, Tetramethylrhodamine, Texas Red, X-rhodamine. Additional fluorophore families include Dyomics series, Atto tec series, coumarins, macromolecular, phycobilliproteins (including phycoerythrins, phycocyanins, and allophycocyanins), green, yellow, red, and other fluorescent proteins, and Quantum Dots. Those skilled in the art will recognize other dyes which may be used within the scope of the invention. This list includes but is not limited to all dyes now known or known in the future which could be used to allow detection of the labeled poly peptide or polynucleotide of the invention.

Electrophoretic Velocity: As used herein, the term “electrophoretic velocity” refers to the velocity of a charged or uncharged target under the influence of an electric field relative to the background electrolyte. Electrophoretic velocity in a capillary system may be a composite measure of electrokinetic velocity and electroosmotic force.

Emission: As used herein, the term “emission” refers to radiation generated by a molecule or particle in processes such as fluorescence, chemiluminescence and elastic or inelastic (e.g., Raman) light scattering.

Emission wavelength: As used herein, the term “emission wavelength” refers to the spectrum of the photons that are released during emission and measured by the detectors used in the analysis instrument. For polyatomic molecules in solution, photon emissions occur over a rather broad spectrum, typically of 100-150 nm. A selected subset of the spectrum is allowed to pass to the detectors by the optical filters used in the instruments. Labels that are detected in the same spectral range are considered to have the same emission wavelength.

Fluid: As used herein the term “fluid” is a medium wherein molecules are suspended and move. It can be aqueous, non-aqueous, or a combination of both that can conduct an electrical current. It may further contain salts, ions, polymers, macromolecules, or other agents that can interact with the polypeptides or polynucleotides and influence their movement.

Fluorescence: As used herein, the term “fluorescence” refers to the photons of energy that are emitted as an excited fluorophore returns to its ground state. The energy of the emitted photon is lower, and therefore of longer wavelength, than the excitation photon.

Fluorescence Burst Duration: As used herein, the term “fluorescence burst duration” refers to the period of time during which an emission event is detected. Fluorescence Intensity: As used herein, the term “fluorescence intensity” refers to the output of a detection system that measures the radiation from a fluorescing sample. It also refers to the number of photons detected per unit time (preferably milliseconds and preferably above a background threshold).

Fluorescence Lifetime: As used herein, the term “fluorescence lifetime” refers to the time required by a population of N excited fluorophores to decrease exponentially to N/e by losing excitation energy through fluorescence and other deactivation pathways.

Fluorescence Polarization: As used herein, the term “fluorescence polarization” refers to the property of fluorescent molecules in solution that are excited with plane-polarized light and emit light back into a fixed plane (i.e., the light remains polarized) if the molecules remain stationary during the excitation and emission cycle of the fluorophore.

Interrogation volume: As used herein the term “interrogation volume” is the space illuminated by the illumination source and “seen,” sensed or otherwise detected by the detectors, through which at least one polypeptide or polynucleotide may traverse.

Label: As used herein, the term “label” refers to a moiety that, when attached to the polypeptide or polynucleotide of the invention, alters detectable parameters of the polypeptide or polynucleotide such as its electromagnetic emission or its electrophoretic velocity. Exemplary labels include but are not limited to fluorophores, chromophores, radioisotopes, spin labels, enzyme labels, chemiluminescent labels, mass tags, charge tags, and charge/mass tags. Such labels allow detection of labeled compounds by a suitable detector. In addition, such labels include components of multi-component labeling schemes, e.g., a system in which a target binds specifically and with high affinity to a detectable binding partner, e.g., a labeled antibody binds to its corresponding antigen. Herein, label and “tag” are used synonymously.

Light Absorption: As used herein, the term “light absorption” refers to the light energy (wavelengths) not reflected by an object or substance.

Light Scattering: As used herein, the term “light scattering” refers to processes by which photons change directions. It includes both elastic light scattering where photons change direction without changing their wavelength and inelastic scattering where the scattered radiation has a different (normally lower) energy from the incident radiation.

Mass tags: As used herein, the term “mass tag” refers to any mass added to the target that serves to distinguish the mass tag+target from the target alone based on detection of the mass, or charge to mass ratio. A mass tag can be a label.

Nucleic acid hybrid: As used herein, the term “nucleic acid hybrid” refers to double-stranded nucleic acid molecule which is made by hybridizing two complementary single-stranded nucleic acid molecules. That is, forming hydrogen bonds between complementary base pairs of the two single-stranded nucleic acid molecules.

PE: As used herein, the term “PE” refers to the dye phycoerythrin.

Sample: As used herein, the term “sample” shall mean a contiguous volume containing at least one detectable polynucleotide and at least one detectable polypeptide. This term shall include, but shall not be limited to, detecting the polynucleotide and polypeptide in one sample run. Also within this definition, the polynucleotide is preferably detected within one hour of detecting the polypeptide from a contiguous volume, more preferably within 15 minutes, still more preferably within 1 minute, and still more preferably 10 microseconds. The term “sample” as described above also refers to the volume that contains only the detectable labels in the case when they are released from the original target molecules, and are analyzed in the released state.

SMD: As used herein, the term “SMD” refers to single molecule detection.

Target: As used herein, the term “target” refers to the entity to be detected in an assay, either a polypeptide or polynucleotide. This term is also known in the art as an analyte.

### Co-Detection of Single Polypeptide and Polynucleotide Molecules

Co-detection of polypeptides and polynucleotides at very low concentrations or at the individual molecule level provides a new powerful tool for medical and biothreat applications. Most infectious organisms contain both of these biomolecules. Detecting the presence of both allows one to not only confirm the presence of the pathogen, but also to assess the course of an infection. For example, a viral pathogen may have a specific gene sequence that identifies the strain of the pathogen and may also code for specific protein toxins or virulence factors that vary with the strain. Detecting both components gives added assurance of the identification and may provide an evaluation of the stage of the disease and prognosis for disease progression. In another example, a pathogen may elicit an immune response resulting in production of antibodies directed against that pathogen. Co-detection of a pathogen's genetic component and the antibody suggests an active infection is in progress, while detecting only the pathogen or only the antibody suggests, respectively, that the infection is recent or has cleared. Similarly, biothreat applications allow for double confirmation of the identity of an organism at very low concentrations. Co-detection of polypeptides and polynucleotides also allows one to assess the risk involved by knowing specifically which organism is present.

Numerous applications for this co-detection technology also exist in biomedical research. The presence of a certain mRNA in a cell does not guarantee that the protein product of the gene is actually made, although this assumption is often made. Many factors including differences in translation efficiency, turnover rates, extracellular expression or compartmentalization, and post-translational modification affect protein levels independent of transcriptional controls. Co-detection would allow one to determine whether mRNA is translated on a cell-by-cell basis and possibly how much is translated. Another application is for comparison of normal and diseased cells. Mutations in genes often result in altered protein translation or degradation. With co-detection one can discover that relationship and, after comparing it to the normal relationship, learn its contribution to the disease state. Another application is for studying the direct interaction of proteins with nucleic acids. Proteins that bind DNA or RNA often have an effect on transcription or translation. Such proteins are often called cofactors, regulatory, or effector molecules. Co-detection technology allows one to identify both the protein and nucleic acid involved in the binding. In addition, proximity assays can be used with co-detection to further characterize the binding.

The advantages of the current technology over previous methods are numerous: Individual molecules can be detected directly without amplification or bead attachment. Multiple parameters of the molecules can be detected simultaneously. The amount of sample required is small. This is important, for example, for forensic biosensor or biomarker applications. The technology also is rapid and requires minimal sample handling. These considerations play a role in situations where changes in molecules are known or suspected to occur during sample preparation. In biological systems, many changes in molecules can occur if hours are needed for sample processing and analysis. Analysis times measured in minutes offer a significant advantage.

The invention described herein overcomes limitations of the previously described technologies. It enables detection of individual polypeptide and polynucleotide molecules without requiring amplification or attachment to a particle. Quantification of molecules only requires counting, which is simple and precise. Multiple parameters can be used to accomplish discrimination between polypeptides and polynucleotides including the electromagnetic characteristics of the labels such as intensity or lifetime of the emission. In addition, the sample can be flowing, or subjected to electrophoretic separation which enables discrimination based on the mobility of the polypeptide and polynucleotide. The method is fast (seconds to minutes) and able to measure large numbers of events. Low protein and nucleic acid concentrations (femtomolar or less) can be detected. Simple and inexpensive instrumentation is used that is composed of a single illumination source, simple detectors and electronics. Data analysis can also be automated using the present invention.

Low protein and nucleic acid concentrations (femtomolar or less) can be detected and molecules can readily be quantitated. The preferred instrumentation is simple and inexpensive with a single illumination source, simple detectors and electronics and the capacity to automate data analysis.

In a preferred embodiment, samples are analyzed in an instrument that consists of a laser (the beam of which may be split into two or more beams), focusing light-collection optics, two single photon detectors, and detection electronics under computer control. Examples of such instruments may be found in U.S. Pat. Nos. 4,793,705, and 5,209,834, each of which is incorporated herein by reference in its entirety. Additional features of the instruments may also be found in U.S. patent application Ser. Nos. 10/720,047, 10/718,194 and 10/720,044, each of which is incorporated herein by reference in its entirety. Two lasers or detectors are not required for co-detection.

This invention provides a method for co-detecting a polynucleotide molecule and a polypeptide molecule contained within one sample comprising (a) moving said polynucleotide molecule and said polypeptide molecule through at least one interrogation volume; and (b) measuring an electromagnetic characteristic of said polynucleotide molecule and measuring an electromagnetic characteristic of said polypeptide molecule, wherein said polynucleotide molecule and polypeptide molecule are co-detected.

In one embodiment, two distinct electromagnetic properties are measured at each interrogation volume. In some cases this can be accomplished though additional analysis of the signal from a single detector as in the case of emission intensity and burst duration. In another example, two detectors can be used at each interrogation volume, such that distinct properties are measured, such as measuring the total photon signal with one set of detectors at each volume and measuring the fluorescence polarization with a second set of detectors.

Co-detection refers to the detection of two or more species within a single sample. In one embodiment, the protein and nucleic acid are detected in a system where the sample fluids are driven by mechanical means to flow past a detector. Examples of mechanical means are pressure (and vacuum) that can be applied to the sample by any controllable fluid delivery system, such as gravity feed, or pump. In another embodiment, the sample is subjected to electrophoresis, such as by placing the sample in an electrophoretic sample channel.

In one embodiment, the interrogation volume can be defined by a dimension of electromagnetic radiation received from the electromagnetic radiation source and/or a range of the electromagnetic radiation detector. In addition, a dimension of the interrogation volume can be adjustable, variable or both adjustable and variable. Alternatively, the interrogation volume can be defined by a wall of a solid material. Alternatively, the interrogation volumes can be defined by a fluid boundary.

In one embodiment, the electromagnetic characteristic is selected from the group consisting of emission wavelength, emission intensity, burst size, burst duration, fluorescence polarity, light scattering, fluorescence lifetime and any combination thereof. In one embodiment, the polynucleotide and polypeptide molecules are both contained within a single sample and the co-detection of the polypeptide and polynucleotide molecules is simultaneous, that is, the electromagnetic characteristics of the two molecules are measured at the precisely same time within the same interrogation volume. In a preferred embodiment, the polypeptide and polynucleotide molecules are both contained within a single sample, and electromagnetic characteristics of polynucleotide and polypeptide molecules are detected sequentially as they traverse the interrogation volume as part of a single sample analysis.

In one aspect of the invention, if the number of molecules of one of the polynucleotide or polypeptide in the sample is known prior to analysis, and a greater number of molecules is detected, the molecules are co-detected, but not discriminated. In a preferred embodiment, the molecules are co-detected and discriminated in the same sample. Discrimination is accomplished by detecting differences in the electromagnetic characteristics of the target molecules. In yet a further aspect, differences in the electrophoretic velocity of the polynucleotide molecules and polypeptide molecules are detected. In one embodiment of the invention, sieving media can be employed to affect the separation of molecules. Thus it will be obvious to one skilled in the art the art that multiple polynucleotides and polypeptides can be distinguished in a mixture by employing a combination of the electromagnetic characteristics of the molecules and the mobility of the molecules. The methods described herein enable at least one polynucleotide and at least one polypeptide molecule to be distinguished individually in a sample comprising multiple molecules. The terms discriminated, detected and distinguished are used interchangeably herein.

In another embodiment, a plurality of polynucleotide molecules and a plurality of polypeptide molecules are co-detected. This plurality includes multiple molecules of the same polynucleotide and multiple molecules of the same polypeptide, single molecules of many different polynucleotides and single molecules of many different polypeptides, multiple molecules of the same polynucleotide and single molecules of many polypeptides, or single molecules of many polynucleotide and multiple molecules of the same polypeptide. The plurality of molecules has a theoretical and function range of limitations. The theoretical and functional lower limit is one molecule of a polynucleotide and one molecule of a polypeptide. The theoretical upper limit depends on the physical limitations of the detectors and the computer software. For example, if 200 molecules can be detected in 10 us (one of the preferred arbitrary time segments with freely selectable channel widths, see below), then 2 million molecules (2×106) are detected per second. In the future, with improved detectors and software, this limit could increase. One skilled in the art will recognize that the upper limit will also depend on how long one analyzes the sample, how much sample is available, and the mode of analysis used, and that these factors will define a functional upper limit. Functional upper limits preferably fall in the range of thousands (103) of molecules, and more preferably fall in the range of hundreds of molecules.

The target molecules of the invention are polypeptides and polynucleotides. In one embodiment of the invention, the polypeptides are equivalent to proteins. The preferred target proteins are functional components of cells such as structural proteins that are components of the cellular cytoskeleton, enzymes, receptors or signaling factors. Polypeptides also include components of proteins such as peptides, epitopes, or binding domains and complexes of proteins such as dimers, trimers, or heteromeric assemblies of discrete protein subunits. Target molecules also include oligopepetides such as hormones or antibiotics.

In one embodiment, polynucleotides are equivalent to nucleic acids. The preferred target nucleic acids are functional components of cells such as DNA contained within chromosomes and messenger, ribosomal or transfer RNA. Polynucleotides further include fragments of DNA or RNA, single stranded or double stranded molecules, or complexes of nucleic acids such as dendrimers or nucleic acid hybrids. In a further embodiment, polynucleotides include oligonucleotides containing naturally occurring and/or modified nucleotides. Further, a polynucleotide can be a polymer comprised of linked nucleotides, and includes DNA or RNA. DNA is a polymer comprised of a phosphodiester backbone composed of monomers of purines and pyrimidines such as adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. RNA is a polymer comprised of a phosphodiester backbone composed of monomers of purines and pyrimidines such as those described for DNA except that uracil is substituted for thymidine. DNA monomers may be linked to each other by their 5′ or 3′ hydroxyl group thereby forming an ester linkage. RNA monomers may be linked to each other by their 5′, 3′ or 2′ hydroxyl group thereby forming an ester linkage. Alternatively, DNA or RNA monomers having a terminal 5′, 3′ or 2′ amino group may be linked to each other by the amino group thereby forming an amide linkage. In some instances, the polymer is a peptide nucleic acid (PNA), or a locked nucleic acid (LNA). In one embodiment, sensitive co-detection of proteins and nucleic acids utilizes molecules which intrinsically produce, or are extrinsically labeled, to emit electromagnetic radiation. Preferred mechanisms of electromagnetic radiation emission include fluorescence, chemiluminescence, and light scattering. In a further embodiment, the molecule may be detected through a combination of an intrinsic property and an extrinsic property. The preferred electromagnetic radiation is fluorescence. In a further embodiment, polypeptides and polynucleotides can be distinguished based on non-electromagnetic characteristics such as mobility. These measurable characteristics may be produced by an intrinsic or extrinsic property of the molecule.

Many naturally occurring units of a polymer are light emitting compounds or quenchers. For instance, nucleotides of native nucleic acid molecules have distinct absorption spectra, e.g., A, G, T, C, and U have absorption maximums at 259 nm, 252 nm, 267 nm, 271 nm, and 258 nm respectively. Modified units which include intrinsic labels may also be incorporated into polymers. A nucleic acid molecule may include, for example, any of the following modified nucleotide units which have the characteristic energy emission patterns of a light emitting compound or a quenching compound: 2,4-dithiouracil, 2,4-Diselenouracil, hypoxanthine, mercaptopurine, 2-aminopurine, and selenopurine.

Protein containing the aromatic amino acids (tryptophan, tyrosine and phenylalanine) may exhibit intrinsic florescence. In addition, there are specific cases of intrinsically fluorescent proteins or protein complexes such as green fluorescent protein (from the jellyfish Aequorea victoria) and phycobiliproteins produced by cyanobacteria and red algae. In one embodiment, the extrinsic property is provided by a label. The methods for labeling the molecule with a means of detection or discrimination are within the ordinary skill in the art. Attaching labels to molecules can employ any known means. In some cases, the method of labeling is non-specific, for example, a method that labels all nucleic acids regardless of their specific nucleotide sequence. In other cases, the labeling is specific, as in where a labeled oligonucleotide binds specifically to a target nucleic acid sequence. Specific and non-specific labeling techniques will be discussed in more detail in the following sections.

Non-specific labeling of nucleic acids generally employs functional groups which label all nucleic acid regardless of the particular nucleotide sequence. One skilled in the art is familiar with various techniques for general labeling of nucleic acids. Methods include: intercalating dyes such as propidium iodide, acridine orange, and Hoechst dyes; labeling the N-7 position of guanine bases (ULYSIS kit); incorporating a chemically reactive nucleotide analog to which a label can be readily attached (Ares kit); incorporation of a biotin containing nucleotide analog for attachment of a strepavidin-bound label. Enzymatic labeling incorporates labeled nucleotide analogs during strand replication. In a preferred embodiment, the non-specific label is directly attached to the target molecule.

Techniques to non-specifically label proteins are also well known to those skilled in the art. For example, the free amine at the N-terminus of the protein, and chemically reactive amino acids, such as lysine, on the surface of proteins can be used to attach a label (Molecular Probes Handbook pp. 7-93). In additions, labels can be added to carbohydrate moieties on glycoproteins (Molecular Probes Handbook pp. 99-112). In some applications, the label can be added to the protein in a two step process where first one member of a high affinity binding system, e.g., biotin is added to the protein, and the label is attached to the other member, e.g., avidin. Isotype specific reagents have also been developed for labeling antibodies (Zenon, Molecular Probes). In a preferred embodiment, the non-specific label is directly attached to the target molecule. In another embodiment, target proteins can be directly labeled by expressing them as a fusion protein with a fluorescent protein such as green fluorescent protein (GFP).

In a preferred embodiment, only specific proteins or nucleic acids within a mixture are labeled. In one embodiment, the label is added indirectly to the target molecules though a labeled binding partner which interacts with the target molecule. Specific labeling can be accomplished by combining a protein or nucleic acid with a labeled binding partner, where the binding partner interacts specifically with the protein or nucleic acid molecule through complementary binding surfaces. Binding forces can be covalent interactions or non-covalent interactions such as hydrophobic interactions, ionic, or hydrogen bonds, van der Waals attraction. Examples of proteins and binding partners includes: proteins and antibodies, antigens and specific antibodies, hormone and hormone receptor, ligands and receptors, and enzymes and enzyme substrates or cofactors. Examples of nucleic acid and binding partners include: polynucleotide and complementary polynucleotide, oligonucleotide and complementary polynucleotide or two complementary oligonucleotides where either the oligonucleotide or polynucleotide can be DNA, RNA, or include analogs or modified bases such as LNAs or PNAs.

In preferred embodiment target polynucleotides can be labeled using an oligonucleotide which specifically hybridizes to the nucleic acid as a primer and nucleic acid synthesis techniques using either polymerase chain reaction (PCR) or run-off transcription (See U.S. patent application Ser. No. 10/718,194 incorporated herein by reference in its entirety). The labels can consist of either fluorescent nucleotide analogs such as ChromaTide dUTP (molecular probes) which are incorporated into the product or reactive nucleotides with functional groups such as aminoallyl dUTP to which fluorescent tags can readily be added.

In a further embodiment, the protein and nucleic acid species are distinguished based on their molecule interaction with one or more binding partners. These interactions may be protein/protein binding, nucleic acid hybridization or protein/nucleic acid binding. Immunoassays based on specific antibody/protein pairs are one example of binding partners. In one case, the interaction may alter the electromagnetic emission properties of either the target protein or nucleic acid species. Examples of this include binding the target molecule to a labeled binding partner or a large species to alter the fluorescence polarization. In another embodiment, the labeled target molecule can be bound to a molecular species containing a fluorescence quencher or a molecular species containing a FRET partner. In a further embodiment, the interaction may alter the mobility of either the protein or nucleic acid species. Examples of this include: mass, charge or mass/charge tags to alter the electophoretic mobility.

One skilled in the art will recognize that labels can be applied before, after, or simultaneously with positioning the molecule into the interrogation fluid. In a preferred embodiment, the molecules are labeled prior to moving through the interrogation volume. In one embodiment, the protein and nucleic acid are labeled in the same reaction mix prior to analysis. In another embodiment, the protein and the nucleic acid are labeled in separate reactions and combined prior to analysis. Once a molecule is detectably labeled, any suitable means of detection that is known in the art can be used.

Labels include dye tags, radioactive tags, charge tags, mass tags, charge/mass tags, light scattering tags, chemiluminescent tags, quantum dots, or beads, polymeric dyes, dyes attached to polymers. Dyes encompass a very large category of compounds that add color to materials or enable generation of luminescent or fluorescent light. A dye may absorb light or emit light at specific wavelengths. A dye may be intercalating, or be noncovalently or covalently bound to a polypeptide or polynucleotide. Dyes themselves may constitute probes as in probes that detect minor groove structures, cruciforms, loops or other conformational elements of molecules. Dyes may include BODIPY and ALEXA dyes, Cy[n] dyes, SYBR dyes, ethidium bromide and related dyes, acridine orange, dimeric cyanine dyes such as TOTO, YOYO, BOBO, TOPRO POPRO, and POPO and their derivatives, bis-benzimide, OliGreen, PicoGreen and related dyes, cyanine dyes, fluorescein, LDS 751, DAPI, AMCA, Cascade Blue, CL-NERF, Dansyl, Dialkylaminocoumarin, 4′,5′-Dichloro-2′,7′-dimethoxyfluorescein, 2′,7′-Dichlorofluorescein, DM-NERF, Eosin, Erythrosin, Fluoroscein, Hydroxycourmarin, Isosulfan blue, Lissamine rhodamine B, Malachite green, Methoxycoumarin, Naphthofluorescein, NBD, Oregon Green, PyMPO, Pyrene, Rhodamine, Rhodol Green, 2′,4′,5′,7′-Tetrabromosulfonefluorescein, Tetramethylrhodamine, Texas Red, X-rhodamine, Dyomic series, Atto tec series, Coumarins, macromolecular, phycobilliproteins (phycoerythrins, phycocyanins, allophycocyanins), green, yellow, red and other fluorescent proteins, metallic crystals and other dyes that interact with or may be attached to molecules. Those skilled in the art will recognize other dyes which may be used within the scope of the invention. This list includes but is not limited to all dyes now known or known in the future which could be used to allow detection of the labeled polypeptide or polynucleotide of the invention. By having fluorescent markers, such as fluorescent molecules, fluorescent conjugated antibodies, or the like, the sample may be irradiated with light absorbed by the fluorescent molecules and the emitted light measured by light measuring devices. Dyes can be employed as the label or produced as a result of a reaction, e.g., an enzymatically catalyzed reaction. Chemiluminescence is the generation of electromagnetic radiation as light by the release of energy as the result of a chemical reaction. While the light can, in principle, be emitted in the ultraviolet, visible or infrared region, those processes emitting visible light are the most common. Types of chemiluminescent reactions include the following: Chemical reactions using synthetic compounds and usually involving a highly oxidized species such as a peroxide are commonly termed chemiluminescent reactions. Light-emitting reactions arising from a living organism, such as the firefly or jellyfish, are commonly termed bioluminescent reactions.

Target molecules can be labeled directly or indirectly with enzyme labels such as alkaline phosphatase, G-6-P dehydrogenase, horseradish peroxidase, luciferase or xanthine oxidase. The labeled target molecules are then combined with chemiluminescent molecules such as luminol, isoluminol, and acridinium esters, prior to entering the interrogation volume. For light scattering, the emission is at the same wavelength as the incident light, but has been dispersed by the molecule itself. Useful light scattering labels include metals, such as gold, silver, platinum, selenium and titanium oxide.

In yet another aspect, the labels affect the electrophoretic analysis and/or separation of target polypeptides or polynucleotides. These labels are referred to as charge/mass tags. Attachment of such a label alters the ratio of charge to translational frictional drag of the target polypeptides or polynucleotides in a manner and to a degree sufficient to affect their electrophoretic velocity and/or separation.

In another embodiment, the label alters the charge, or the mass, or a combination of charge and mass. The charge/mass tag bound to a polypeptide or polynucleotide can be discriminated from the unbound polypeptide or polynucleotide or unbound tag by virtue of spatial differences in their behavior in an electric field, and by virtue of velocity differences in their behavior in an electric field.

Direct coupling attaches the binding partners to the charge/mass tags. Indirect coupling can be accomplished by several methods. The binding partners may be coupled to one member of a high affinity binding system, e.g., biotin, and the tags attached to the other member, e.g., avidin. For binding partners which are antibodies, one may also use second stage antibodies that recognize species-specific epitopes of the antibodies, e.g., anti-mouse lg, anti-rat lg, and the like. Indirect coupling methods allow the use of a single charge/mass tag, e.g., antibody, avidin, and the like, with a variety of binding partners. Those of skill in the art will recognize several methods for providing labeled secondary antibodies against primary antibodies.

Especially useful binding partners are antibodies specific for the target. Whole antibodies may be used, or fragments, e.g., Fab, F(ab′)₂, and light or heavy chain fragments. Such antibodies may be polyclonal or monoclonal and are generally commercially available or alternatively, readily produced by techniques known to those skilled in the art. Antibodies selected for use will have a low level of non-specific binding. In a further embodiment, binding partners that are oligonucleotides or polynucleotides which specifically bind to the target are useful reagents.

To accurately detect a labeled target molecule, the labeled molecule must be distinguished from unbound label. Many ways to accomplish this are familiar to those skilled in the art. For example, in heterogeneous assays, unbound label is separated from labeled molecules prior to analysis. In a preferred embodiment, the sample, including unbound label, is analyzed by a combination of electrophoresis and individual molecule fluorescence detection. In this case, electrophoretic conditions are chosen which provide distinct velocities for the labeled molecule versus the unbound label.

In one embodiment, the protein and nucleic acid are detected in a system where the sample fluids are driven by mechanical means to flow past a detector. Examples of mechanical means are pressure (and vacuum) that can be applied to the sample by any controllable fluid delivery system, such as gravity feed, or pump. Generally these methods demonstrate parabolic flow and all molecules move at a constant velocity as detected in suitable detection systems.

In another embodiment, the sample is subjected to electrophoresis, such as by placing the sample in an electrophoretic sample channel. Mobility of polypeptides or polynucleotides within the sample fluid varies with the properties of the polypeptide or polynucleotide. The velocity of movement produced by electrokinetic force is determined by the relative charge and mass of the individual polypeptide or polynucleotide and the electroosmotic force. Movement of a polypeptide or polynucleotide can be altered by the type of label that has been attached to the molecule, such as a charge/mass tag. The electrophoretic velocity of the molecules can also be altered by changes in the electroosmotic flow. In an additional embodiment, when two or more polypeptides or polynucleotides are present, at least one may move through at least one interrogation volume in a direction opposite to that of the other molecule.

In one embodiment, the electrophoretic velocity of each detectably labeled molecule is determined. Based on the determination of the electrophoretic velocity of each detectably labeled molecule, individual molecules in a sample comprising multiple molecules can be distinguished. Any electrophoretic separation technique together with immunoassay or nucleic acid hybridization labeling can be, in principle, adapted for use in the context of the present invention. The electrophoretic velocity of the molecule can be modified through additional forces such as pressure or vacuum, surface tension or gravitational flow.

In a preferred embodiment, the sample comprises a buffer. While any suitable buffer can be used, the preferable buffer has low fluorescence background, is inert to the detectably labeled molecule, can maintain the working pH and has suitable ionic strength for electrophoresis. The buffer concentration can be any suitable concentration, such as in the range from 1-200 mM. Preferably, the buffer is selected from the group consisting of Tris, Gly-Gly, bicine, tricine, MES, MOPS and AMP. Preferred buffers include: Tris/borate, Tris/glycine and Tris/HCl.

For some applications, the buffer desirably further comprises a sieving matrix for use in this method. While any suitable sieving matrix can be used, desirably the sieving matrix has low fluorescence background and can specifically provide size-dependent retardation of detectably labeled molecules. The sieving matrix can be present in any suitable concentration; from about 0.1% to about 10% is preferred. Any suitable molecular weight can be used; from about 100,000 to about 10 million is preferred. Examples of sieving matrixes include poly(ethylene oxide) (PEO), poly(vinylpyrrolidine) (PVP), linear polyacrylamide and derivatives (LPA), hydroxypropylmethylcellulose (HPMC) and hydroxyethylcellulose (HEC), all of which are soluble in water and have exceptionally low viscosity in dilute concentration (0.3% wt/vol). In addition, these polymer solutions are all below their entanglement threshold and are easy to prepare, filter and fill into capillary tubes.

Electrokinetic force can be combined with other motive forces such as pressure, vacuum, gravity, surface tension, and centrifugal to discriminate between protein and nucleic acid molecules. In one embodiment, these forces can be chosen so as to have differential effects on different molecules within a sample so that when two or more molecules are present, at least one molecule moves through the interrogation volume with a velocity that differs from the other molecule(s). The velocities of the target molecules can be aligned with the fluid flow or at least one molecule can move antiparallel to the fluid flow. In another embodiment, at least one molecule has an antiparallel velocity exceeding the velocity of the fluid flow. In another embodiment, at least one molecule is in motion perpendicular to the fluid flow. In another embodiment, at least one molecule is in motion with a combination of motions that are antiparallel and perpendicular to the fluid flow.

Several approaches can be used to distinguish between labeled (or intrinsically fluorescent) proteins and nucleic acids. In one embodiment, the protein and nucleic acid are distinguished based on electromagnetic properties of their respectively labels. In another embodiment, physical properties such as velocity in an electric field are used to distinguish between the protein and nucleic acid.

In one embodiment, electromagnetic emission refers to the release of photons from a molecule in response to a stimulus. In the case of fluorescent emission, the stimulus is absorbed light. In the case of chemiluminescence and bioluminescence, the stimulus is a chemical reaction or biochemical reaction.

For elastic light scattering, the emission is at the same wavelength as the incident light, but has been dispersed by the molecule itself. In other cases, there is a scattered light is of a different wavelength than the incident light. For example, when nano-sized metal colloid particle are illuminated with a standard while light source the scattering produces intense monochromatic light.

Light, particularly light in the ultra-violet, visible or infrared range, is the preferred electromagnetic radiation to detect. The detectors of the instrument are capable of capturing the amplitude and duration of photon bursts from fluorescent particles and converting them to electronic signals. Detection devices such as CCD cameras, video input module cameras, and Streak cameras can be used to produce images with contiguous signals. In another embodiment, devices such as a bolometer, a photodiode, a photodiode array, avalanche photodiodes, and photomultipliers which produce sequential signals can be used. In a preferred embodiment, avalanche photodiodes are used for the very sensitive detection of photons. Using specific optics between the interrogation volume and the detector, several distinct characteristics of the emitted electromagnetic radiation can be detected including: emission wavelength, emission intensity, burst size, burst duration, fluorescence lifetime, and fluorescence polarization. It will be obvious to one skilled in the art that one or more detectors can be configured at each interrogation volume and that the individual detectors may be configured to detect any of the characteristics of the emitted electromagnetic radiation listed above.

The preferred illumination sources are continuous wave lasers for wavelengths of >200-1000 nm. These illumination sources have the advantage of being small, durable and relatively inexpensive. In addition, they generally have the capacity to generate larger fluorescent signal than other light sources. Specific examples of suitable lasers include: lasers of the argon, krypton, helium-neon, helium-cadmium types as well as tunable diode lasers (red to infrared regions), each with the possibility of frequency doubling. The lasers provide continuous illumination with no accessory electronic or mechanical devices such as shutters, to interrupt their illumination. Light emitting diodes (LEDs) are another low-cost, high reliability illumination source. Recent advances in ultra-bright LEDs coupled with dyes with high absorption cross-section and quantum yield, support their applicability to single molecule detection. Such lasers could be used alone or in combination with other light sources such as mercury arc lamps, elemental arc lamps, halogen lamps, arc discharges, plasma discharges, light-emitting diodes, or combination of these. The optimal laser intensity depends on the photo bleaching characteristics of the individual dyes and the length of time required to traverse the interrogation volume (including the speed of the particle, the distance between interrogation volumes and the size of the interrogation volumes). To obtain a maximal signal, it is desirable to illuminate the sample at the highest laser intensity which will not result in photo bleaching a high percentage of the dyes. The preferred laser intensity is one such that no more that 5% of the dyes are bleached by the time the particle has traversed the final interrogation volume.

Alternatively, pulsed lasers can be used as illumination sources. Pulsed lasers together with time-gated detectors can be used for determining the fluorescence lifetime of labeled polypeptides and polynucleotides as one option for discrimination and co-detection. In the case of fluorescent emissions, the photon signal detected depends both on the wavelength spectra of the fluorescent emission and the filters used with the detectors in the instrument. Therefore, labels with different but overlapping emission spectra may appear indistinguishable if the filter range encompasses both spectra.

In addition, several distinct characteristics of electromagnetic radiation can be measured including properties such as emission intensity, burst size, burst duration, fluorescence lifetime or fluorescence polarization. Fluorescence intensity is quantitatively dependent on the fluorescence quantum yield of the dye, the excitation source intensity, polarity and wavelength, and the detection efficiency of the instrument. It is also affected by the components of the solution, including solvents, ions (such as those that determine pH) and the concentration of the dye, Dye intensity can change if the molecule is exposed to a light source that causes photobleaching.

In one embodiment, both the protein and nucleic acid are labeled with labels having indistinguishable emission wavelengths. It is clear to one skilled in the art that fluorescent emission spectra from typical dyes range over approximately 100-150 nm. Therefore a given dye can be detected at a variety of different wavelengths In this case, the protein and nucleic acid can be distinguished by detection of other electromagnetic properties such as emission intensity, burst size, burst duration, fluorescence lifetime or fluorescence polarization. In the case of fluorescent emissions, the photon signal detected depends both on the wavelength spectra of the fluorescent emission and the optics used with the detectors in the instrument. Alternatively, the protein and nucleic acid can be distinguished based on non-electromagnetic properties such as mobility including electrophoretic velocity.

In one embodiment, the electromagnetic labels on the protein and nucleic acid molecules cannot be distinguished on any of the electromagnetic characteristics listed above. In these cases, other methods can be used to co-detect the protein and the nucleic acid. For example, an additional binding interaction can introduce a second label to alter the electromagnetic properties of the target. Examples of such labels include fluorescence quencher and FRET pairs. In another embodiment, a protein and nucleic acid can be co-detected by differences in fluorescent intensity resulting from different numbers of labels bound to the two species (See U.S. patent application Ser. No. 10/720,044 incorporated herein by reference in its entirety). In other cases, the protein and nucleic acid can be distinguished based on their mobility. This can be the result of intrinsic mobility differences of the molecules in an electric field or due to extrinsic charge/mass tags.

In one embodiment, the polynucleotide molecule can be labeled at a high density, with a range of 50 labels/molecule, and the polypeptide molecule can be labeled with a range of 5 labels/molecule. In this case, the polynucleotide can be distinguished from the polypeptide based on the fluorescence intensity. In one embodiment, the protein and nucleic are distinguished based on different electrophoretic velocities. As discussed above, the mobility of molecules within the sample fluid varies with the properties of the molecule. Specifically, the velocity of movement produced by electrokinetic force is determined by the relative charge and mass of the individual molecule. In some cases, electrophoretic conditions can be readily identified where the protein and nucleic acid molecules have different velocities. In a further embodiment, a mass/charge tag may be added to either the protein or nucleic acid to alter its electrophoretic velocity and allow the two species to be distinguished. It is clear to one skilled in the art that multiplexing to characterize multiple species of protein and nucleic acid in a single sample can be accomplished by combining both differences in electrophoretic velocity and differences in electromagnetic emission characteristics to generate a unique pattern for each species in the sample.

The methods described herein allow individual polypeptides or polynucleotides to be enumerated as they pass through the interrogation volumes and thereby elucidating the concentrations of the target protein and nucleic acid molecules in the sample. Therefore, the concentration of a test sample can be determined without a reference to a standard curve by counting the detected molecules passing through the interrogation volume. The concentration of the sample can be determined from the number of molecules counted and the volume of sample passing though the interrogation volume in a set length of time. In the case where the interrogation volume encompasses the entire cross-section of the sample stream; only the number of molecules counted and the volume passing through a cross-section of the sample stream in a set length of time are needed to calculate the concentration the sample. In another embodiment, the concentration of a polypeptide or polynucleotide in a sample can be determined by interpolating from a standard curve generated with a control sample of known concentration. In a further embodiment, the concentration of a polypeptide or polynucleotide in a sample can be determined by comparing the measured polynucleotide and polypeptide molecules to an internal molecular standard.

In one aspect of the invention, the velocity of the molecules is determined by the time needed to pass through or between two discrete interrogation volumes and the polypeptide and polynucleotide molecules can be distinguished by differences in velocity. Specifically, the sample comprises polynucleotide molecules and polypeptide molecules, and co-detection comprises: measuring an electromagnetic characteristic of a polynucleotide molecule as the polynucleotide molecule interacts with an excitation source within a first interrogation volume; and measuring the electromagnetic characteristic of the polynucleotide molecule as the polynucleotide molecule interacts with an excitation source within a second interrogation volume; then comparing the measured first and measured second electromagnetic characteristics of the polynucleotide; in addition measuring an electromagnetic characteristic of a polypeptide molecule as the polypeptide molecule interacts with an excitation source within a first interrogation volume; measuring the electromagnetic characteristic of the polypeptide molecule as the polypeptide molecule interacts with an excitation source within a second interrogation volume; and comparing the measured first and measured second electromagnetic characteristics of the polypeptide.

By comparing the electromagnetic characteristics measured at the two detectors, the velocities of the labeled components of the sample are determined and the polypeptide molecule and polynucleotide molecule co-detected. In one embodiment, the analysis of data from protein and nucleic acid detection includes cross-correlation. In another embodiment, photon signals are cross-correlated directly. In this case the fluorescent signals (photons) emitted by the sample which come from at least two interrogation volumes are detected by at least two detectors. The signals respectively detected in the detectors are cut into uniform arbitrary, time segments with freely selectable time channel widths. Preferred channel widths (bins) are in the range of 10 us to 5 ms. The number of signals contained in each segment is established. For each time segment from the first detection unit, a cross-correlation analysis with at each segment of the second detection unit is performed. At least one statistical analysis of the results of the coincidence analysis is performed, and/or the results are subjected to a threshold analysis. Said statistical analysis or at least one combination of several statistical analyses is evaluated for the presence of molecules. In this way, a molecule is discriminated from stochastic and background noise based on the presence of correlated signal(s) in at least two detector channels.

In a preferred embodiment, the detected signal is first analyzed to determine the noise level and signals are selected above a threshold prior to cross correlating the data. In one embodiment, the noise level is determined by averaging the signal over a large number of bins. In other embodiments, the background level is determined from the mean noise level, or the root-mean-square noise. In other cases, a typical noise value is chosen or a statistical value. In most cases, the noise is expected to follow a Poisson distribution.

A threshold value is determined to discriminate true signals (peaks, bumps, molecules) from noise. Care must be taken for choosing a threshold value such that the number of false positive signals from random noise is minimized while the number of true signals which are rejected is minimized. Methods for choosing a threshold value include: determining a fixed value above the noise level and calculating a threshold value based on the distribution of the noise signal. In a preferred embodiment, the threshold is set at a fixed number of standard deviations above the background level. Assuming a Poisson distribution of the noise, using this method one can estimate the number of false positive signals over the time course of the experiment. Then cross-correlation analysis is performed on the signals identified from the two detectors.

The time-offset of the cross-correlated signals provides the transit time between the corresponding detectors and therefore based on the distance between the detectors, the electrophoretic velocity of the polypeptide or polynucleotide is determined.

In some cases, a polypeptide or polynucleotide is detected by the fact that the time off-set corresponds to a known time offset. In other cases, a polypeptide or polynucleotide is detected via unknown offset which is determined via population distribution. Therefore, polypeptide or polynucleotides in a mixture labeled with labels with identical electromagnetic properties can be distinguished based on their electrophoretic velocity.

In another embodiment, labels with different electromagnetic characteristics may be used to discriminate between a protein and a nucleic acid. Further, it will be recognized by those of skill in the art that employing labels with different electromagnetic characteristics together with measuring electrophoretic mobility will allow for the increased detection and discrimination of polypeptides or polynucleotides.

In a further embodiment, the cross-correlation analysis can be performed on data from more than two detectors, such as 3, 4, 5, and 6 detectors, or any other desired number that are distinct either in relative location of the interrogation volume or in the wavelength detected. In this case, the cross-correlation analysis can performed on data from any combination of detectors that are distinct. For example, in a case where three detectors, each detecting a distinct wavelength emission (R, G & B) are at each of two interrogation volumes (1 & 2), R1 is correlated with R2, G1 is correlated with G2 and B1 is correlated with B2; resulting in time offsets for molecules with wavelength emission detected by the individual detectors. Other combinations of cross-correlation analysis can also be performed, such as overlapping sets where R1 is correlated with G1; R1 is correlated with B1 and G1 is correlated with B1. Results of these cross-correlation analyses would indicate the frequency of double-labeled polypeptides or polynucleotides. Different combinations of cross-correlation analyses can be used with one another to distinguish molecules based on velocity and labeling (color). In addition, using multiple pairs of cross-correlation analysis will result in more accurate determination of the properties of the individual polypeptide or polynucleotides with in the mixture. In a further embodiment, analysis methods are employed wherein cross-correlation analysis is performed on data from detectors in any combinations of locations and/or wavelengths that are distinct.

Methods for attaching labels to polypeptide molecules and polynucleotide molecules described in detail above in the broad embodiment can also be used in this case. In one embodiment the labels are released from the polynucleotide and polypeptide molecules prior to the analysis. Methods of release are well known to those skilled in the art. For example, labels attached by protein/protein interactions can typically be disrupted using agents such as low pH solutions, such as 100 mM glycine-HCl pH 2.8), by addition of chaotropic agents such as urea or detergents. Labels attached by nucleic acid hybridization can be released using low ionic strength solutions and/or agents such as increased temperature. Those skilled in the art would recognize these and other agents to be effective in removing labels attached to polynucleotide or polypeptide molecules. In addition, labels can be removed by enzymatic cleavage of the molecule/target complex.

Also well know in the art are methods for removing, or rendering non-detectable, excess label not bound to target molecules. These methods include physical separation such as washing to remove unbound label not attached to a surface, or filtering to remove the smaller unbound label from a large target/label complex. In addition, there are methods of rendering excess label non-detectable such as binding a fluorescent label to a complementary molecule which contains a fluorescence quencher. Those of skill in the art will recognize other methods of rendering excess labels non-detectable.

In a further embodiment, the number of labels attached to the polynucleotide and polypeptide molecules and subsequently released and detected is proportional to the number of polynucleotide and polypeptide molecules in the original sample. The relationship between the labels counted and the target molecules can be a linear or non-linear correlation. This relationship can be predefined or determined as a result of analyzing the sample.

### EXAMPLES

Without further elaboration, it is believed that one skilled in the art can, using the preceding description, utilize the present invention to its fullest extent. The following specific examples are offered by way of illustration and not by way of limiting the remaining disclosure.

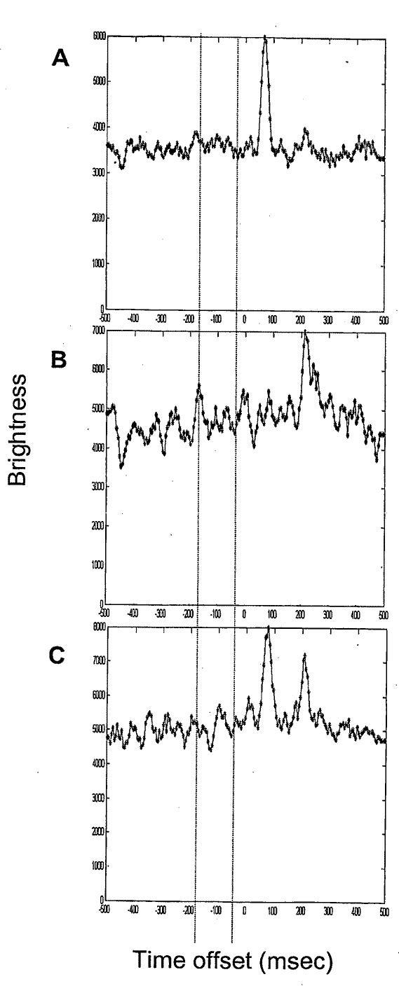

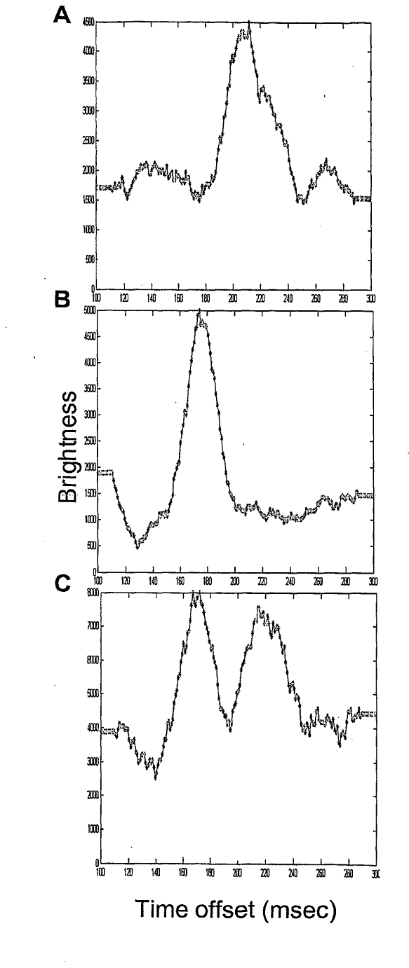

A fragment of DNA, a protein, and a protein complex bound to a nucleic acid were subjected to electrophoresis. Data was analyzed in each of the following examples by analyzing grouped adjacent 1 msec detection blocks derived from molecules using the instrument software. Molecule-derived photon bursts were then cross-correlated to determine their electrophoretic velocities (time offset for detection at the two detectors). Examples of the histogram plots of the molecule cross correlations are shown in FIGS. 1-7.

### Example 1

### Discrimination of a Protein and Nucleic Acid Directed Labeled with the Same Fluorescent Dye—Electrophoresis with Linear Polyacrlyamide

Samples of Alexa Fluor® 647-labeled IgG and 1.1 kb PCR product were prepared in 18 mM tris, 18 mM glycine, pH 8.6 with 0.2% linear polyacrylamide (LPA, 5,000,000-6,000,000 mw), 0.01% sodium dodecyl sulfate and 1 μg/ml each bovine serum albumin, Ficoll®, and polyvinylpyrrolidone. Samples were pumped into the SMD capillary, the pump was stopped, and an electric field was applied (300 V/cm). Cross-correlation of the molecules was determined as a function of time offset. One minute data sets were collected and analyzed.

Examples of the histogram plots of the molecule cross correlations are shown in FIG. 1. A. 26 fM Alexa Fluor 647 labeled IgG (cross-correlation peak at 75 msec). B. 10 fM Alexa Fluor labeled PCR product (peak at 220 msec). C. 13 fM Alexa Fluor 647 labeled IgG and 5 fM Alexa Fluor labeled PCR product (peaks at 75 and 215 msec).

### Example 2

### Discrimination of a Protein and Nucleic Acid Directed Labeled with the Same Fluorescent Dye—Electrophoresis

SMD electrophoretic separation of a protein and a nucleic acid. Samples of Alexa Fluor® 647 labeled IgG and 1.1 kb PCR product were prepared in 18 mM tris, 18 mM glycine, pH 8.6 with 0.01% sodium dodecyl sulfate and 1 μg/ml each bovine serum albumin, Ficoll®, and polyvinylpyrrolidone. Samples were pumped into the SMD capillary, the pump was stopped, and an electric field was applied (150 V/cm). Cross-correlation of the molecules between channels 1 and 2 was determined as a function of time offset. One minute data sets were collected and analyzed. a: 52 fM Alexa Fluor 647 labeled IgG (cross-correlation peak at 210 msec). b: 20 fM Alexa Fluor labeled PCR product (peak at 175 msec). c: 26 fM Alexa Fluor 647 labeled IgG and 10 fM Alexa Fluor labeled PCR product (peaks at 170 and 215 msec).

### Example 3

### Differences in the Characteristic Intensity of Fluorescence Emission were Used to Distinguish a Protein Complex and a Nucleic Acid within a Mixture

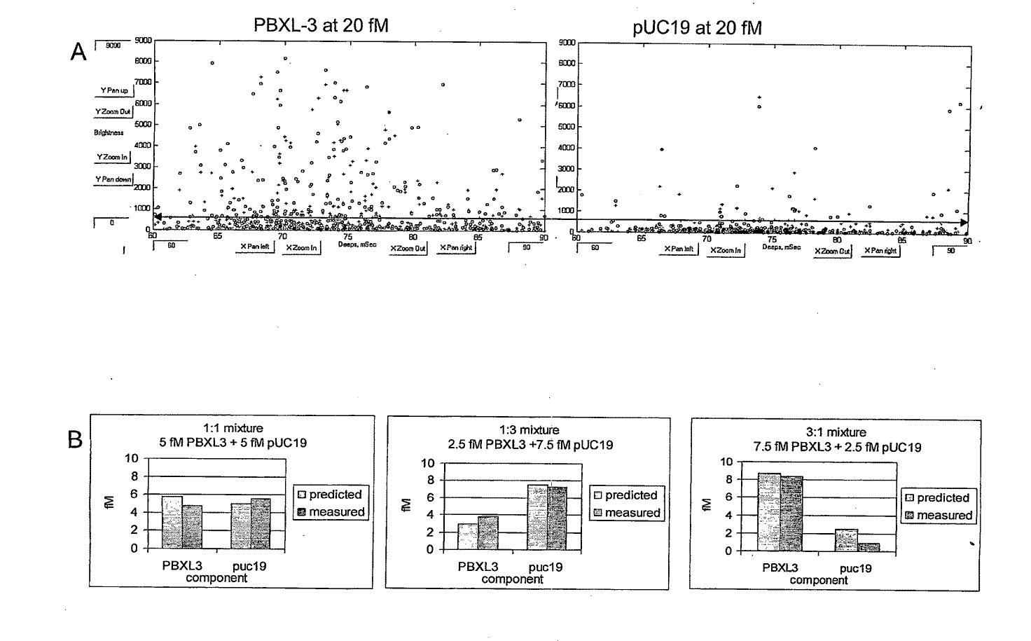

This example also demonstrates that the concentration of sample components can be determined by comparing the counts detected to a standard curve. The protein, PBXL-3, emits at a generally high intensity, and the nucleic acid, linearized pUC19, emits at a generally lower intensity. PBXL-3 is an intrinsically fluorescent protein complex. The pUC19 DNA was labeled with Alexa Fluor® 647 following the protocol of a ULYSIS® nucleic acid labeling kit (Molecular Probes, Inc. Eugene, Oreg.). PBLX-3-strepavidin was purchased from Martek Biosciences Corp. (Columbia, Md.). Phosphate Buffered Solution (PBS) (10 mM NaPO₄, 150 mM NaCl, pH 7.2) was supplemented with 0.01% Casein Acid Hydrolysate and used to make dilution series (2.5, 5, 7.5, 10 and 20 fM) of protein alone, nucleic acid alone or mixtures of both. Samples were moved through the analyzer by pumping at 1 μL/min for 4 min. The data is shown in FIG. 3.

Brightness windows were separated at the intensity of 500 photons. This separation intensity was determined from the plots of intensity for PBXL-3 alone and pUC19 alone at 20 fM (FIG. 3A). Standard curves were plotted for the protein and nucleic acid in both brightness windows and the slopes of the curves were determined. The protein and nucleic acid showed distinct patterns of fluorescence intensities in these plots allowing for discrimination between them. Furthermore, the number of molecules detected in the mixtures of PBXL-3 and pUC19 were used to calculate the concentrations of each component based on the slopes of the standard curves. The measured concentrations for the protein and nucleic acid were compared to the predicted values in FIG. 3B. Counting molecules determined concentrations equivalent to concentrations determined by macro spectrophotometric measurement of undiluted stock solutions.

### Example 4

### The Mobility of a Protein Complex was Shifted after Binding to a Mobility Label Consisting of a Nucleic Acid Molecule, a Method which can be Used to Distinguish Protein and Nucleic Acid by Electrophoretic Mobility

Streptavidin labeled PBXL-3 (PBXL-3/SA) (Martek Biosciences Corp., Columbia, Md.) was combined with a biotin-labeled 1 kb PCR fragment (b-NA) to demonstrate detection of binding interactions. Equi-molar concentrations of PBSL-3/SA and b-NA (800 pM) were incubated at room temperature for at least 1 hr in 10 mM Tris, 0.5 mM EDTA pH 8.1 with 0.1% casein hydrolysate as a carrier. Control incubations were PBXL-3/SA alone and PBXL-3/SA with the same 1 kb fragment without biotin (NA). Following the incubation, samples were diluted 10,000× to final concentration of 8 fM in 2 mM Tris, 0.1 mM EDTA pH 8.1. Samples were loaded into an SMD similar to that disclosed in U.S. Pat. No. 4,793,705 and subjected to electrophoresis for 4 min.

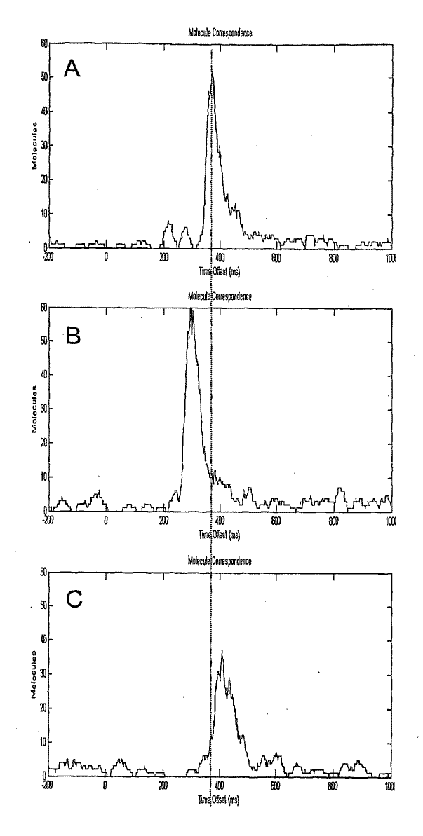

Examples of the histogram plots of the molecule cross correlations are shown in FIG. 4. In the absence of nucleic add, the organelle (PBXL-3/SA) migrated as a peak at 368 (A). Bound to the nucleic acid, it migrated more slowly, as seen by the shift of the peak to 294 ms (B). The shift only occurred when the nucleic acid was bound to the organelle, since its presence (without the biotin tag) in the reaction resulted in the organelle migrating as a peak at 409 ms (C). The standard deviation of electrophoretic velocities is 15 ms.

### Example 5

### Utility in Multiple Apparatus

### Example 5A

This example describes how a bead-based florescence detection instrument can be used for the co-detection of proteins and nucleic acids. Proteins and nucleic acid can be co-detected in a bead-based fluorescence detection instrument. To accomplish this one color of beads can be labeled with an antibody specific for the target protein and beads with a distinct spectral profile can be labeled with an oligonucleotide specific for the target nucleic acid. In addition, labeled detection reagents specific for the target protein and nucleic acid (an antibody and an oligonucleotide, respectively) labeled with PE can be constructed. Sample containing the target protein and nucleic acid can be incubated with beads and washed then incubated with the detection reagents and washed. The sample can then be run on the instrument with two illumination sources, one which excites the bead dyes and another for the detection reagent (PE). For each particle, the emission from the bead dyes will identify the target and the emission from the detection reagent can be used for quantitation of the target.

### Example 5B

This example describes how a capillary electrophoresis instrument with laser induced fluorescence detection can be used for the co-detection of proteins and nucleic acids. Samples containing target protein and nucleic acid molecules labeled with indistinguishable labels (and containing ˜10 nM of each target molecule) can be electrophoresed though a capillary. Laser induced fluorescence was detected using an avalanche photodiode detector. With different electrophoretic velocities, the protein and nucleic acid will reach the detector at different times, and therefore can be detected based on their characteristic migration time.

### Example 5C

This example describes how single molecule electrophoresis instrument with pulsed laser and time-gated detection can be used for the co-detection of proteins and nucleic acids. For example the target protein can be labeled with a dye such as Alexa 647 (with a short fluorescence lifetime (nanoseconds)) and the target nucleic acid with a long lifetime dye (microseconds) such as those described by Herman et al., (2001) and Maliwal et al., (2001)). Sample containing the target molecules can be pumped (if using only lifetime discrimination) or electrophoresed (if using lifetime and velocity discrimination) through the interrogation volume. In this instrument, a pulsed laser can be used as an illumination source. Time-gated avalanche photodiode detectors can be used to detect photons from individual molecules which traverse the interrogation volume. Time-resolved data can then be analyzed using methods such as maximum likelihood estimators. By comparing the measured lifetime measurements with know standards the protein and nucleic acid can be co-detected.

### Example 5D

This example describes how a single molecule electrophoresis instrument with CCD detection can be used for the co-detection of proteins and nucleic acids. The electrophoretic mobility differences of individual proteins and nucleic acids labeled with dyes emitting at the same wavelength can also be detected using imaging with a CCD camera. In this case, the illumination laser is configured to illuminate a plane within the volume electrophoretic path of the laser. Labeled protein and nucleic acid molecules can be electrophoresed through a capillary. The fluorescent signal can be detected with a CCD camera where a sequence of consecutive images are taken and the velocity of the molecule calculated by the distance moved per unit time.

### Example 5E

This example describes how a fluorescence correlation spectroscopy together with electrophoresis can be used for the co-detection of proteins and nucleic acids. Samples containing target protein and nucleic acid molecules labeled with indistinguishable labels (and containing ˜1 nM of each target molecule) can be electrophoresed though a capillary with or without sieving medium. In this example, detection is accomplished in a single interrogation volume using a confocal illumination and detection geometry. Data from the avalanche photodiode detectors can be collected in successive 500 us bins and autocorrelation analysis performed to determine the average transit time of molecules through the interrogation volume. Based on their characteristic electrophoretic velocities protein and nucleic acid molecules can be co-detected.

### Example 5F

This example describes how a two beam fluorescence cross-correlations spectroscopy together with electrophoresis can be used for the co-detection of proteins and nucleic acids. Samples containing target protein and nucleic acid molecules labeled with indistinguishable labels (and containing ˜1 nM of each target molecule) can be electrophoresed though a capillary. In this example, detection can be accomplished in two closely spaced interrogation volumes using a confocal illumination and detection geometry. Data from the two avalanche photodiode detectors can be collected in successive 500 us bins and cross-correlation analysis performed to determine the average transit time between the interrogation volumes for each molecule. Based on their characteristic electrophoretic velocities protein and nucleic acid molecules can be co-detected.

### Example 5G

This example describes how a protein and a nucleic acid can be co-detected using an instrument where the molecule is pulled, drawn or otherwise passes through a pore and individual units of the biopolymer are detected sequentially. For example, the target nucleic acid can be labeled with a fluorescent tag on all of the adenine residues, resulting in multiply substituted molecules. The N-terminus of the target protein can be labeled with the same tag. The samples can then be combined and analyzed by traversing individual molecules through nanopores and measuring the fluorescent signals. Only a single photon burst will be detected for each protein molecule, which passed by the detection station. In contrast, multiple photon bursts will be detected for each nucleic acid as each labeled adenosine passes through the pore. Therefore, based on the relative number of fluorescent labels detected per molecule, the protein and nucleic acid can be co-detected.

### Example 6

### Discrimination of a Protein and Nucleic Acid is Based on Detection of their Respective Labels after Release from the Original Target Molecule

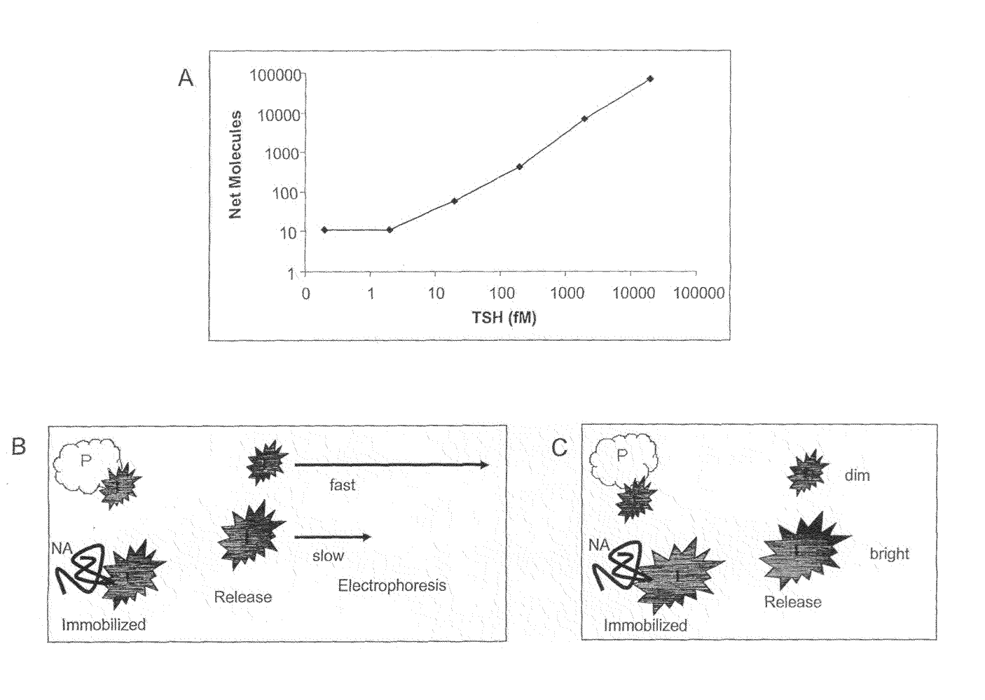

Biotinylated anti-thyroid stimulating hormone (TSH) antibody was immobilized on a streptavidin-coated 96 well plate, and the excess unbound antibody was washed away. TSH antigen and Alexa Fluor®647 labeled anti-TSH antibody were added to the wells in phosphate buffered saline with 1% bovine serum albumin and 0.1% Tween® 20. The plate was incubated with agitation. The liquid was removed by aspiration, and the wells were washed three times. The Alexa Fluor 647 labeled antibody was dissociated from the TSH sandwich by incubation with 0.1 M glycine-HCl, pH 2.8. The free Alexa Fluor 647 labeled antibody was collected, diluted and analyzed by SMD. The linear relationship between released label and the original target molecule concentration is seen in FIG. 5A.

It is obvious to one skilled in the art that similar methods are available for labeling and release of labels from nucleic acids. Matray et al. teaches methods for labeling and releasing labels from both proteins and nucleic acids (Matray, 2004). One skilled in the art will also recognize that separation and discrimination of a mixture of labels released from the target proteins and nucleic acids is essentially the same as for the original targets, such as was demonstrated in Examples 1 and 2. FIGS. 5B and C shows two possible ways to distinguish two released labels.

### Example 7

### Fluorescence Resonance Energy Transfer (FRET) Examples

### Example 7A

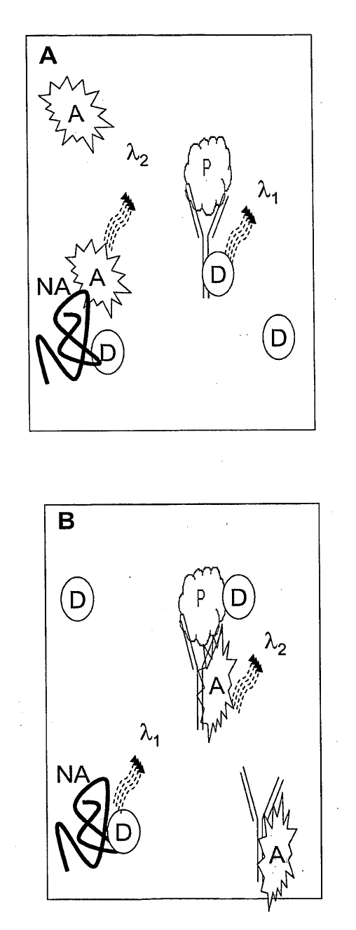

This example describes co-detection of a protein and nucleic acid from an organism using two oligonucleotides containing a FRET donor and acceptor pair to distinguish the protein (P) from the nucleic acid (NA). An oligonucleotide specific for the target nucleic acid is labeled with a donor component (D) of a FRET pair and a primary antibody to the target protein can be labeled with the same label. A second oligonucleotide probe which hybridizes near the first probe can be labeled with a FRET acceptor (A). The oligonucleotides and antibody can be incubated with a sample containing the target protein and nucleic acid. The sample can then be pumped through an interrogation volume and the fluorescent signal measured by two detectors (with filters to discriminate between the donor and acceptor fluorescence). Based on the relative signals from the two detectors, the protein and nucleic acid can be co-detected, FIG. 6A.

### Example 7B

This example describes the co-detection of a protein and nucleic acid using an antibody labeled with a FRET acceptor to distinguish the protein from the nucleic acid. Samples containing target protein and nucleic acid molecules can be labeled with indistinguishable labels (the donor component of a FRET pair) and an antibody specific for the target protein is labeled with FRET acceptor molecules. The antibody can be incubated with a sample containing the target protein and nucleic acid. The sample can then be pumped through an interrogation volume and the fluorescent signal measured by two detectors (with filters to discriminate between the donor and acceptor fluorescence). Based on the relative signals from the two detectors, the protein and nucleic acid can be co-detected, FIG. 6B.

### Example 8

### Discrimination of a Protein and Nucleic Acid Based on Molecular Mobility in a Two Dimensional Separation System

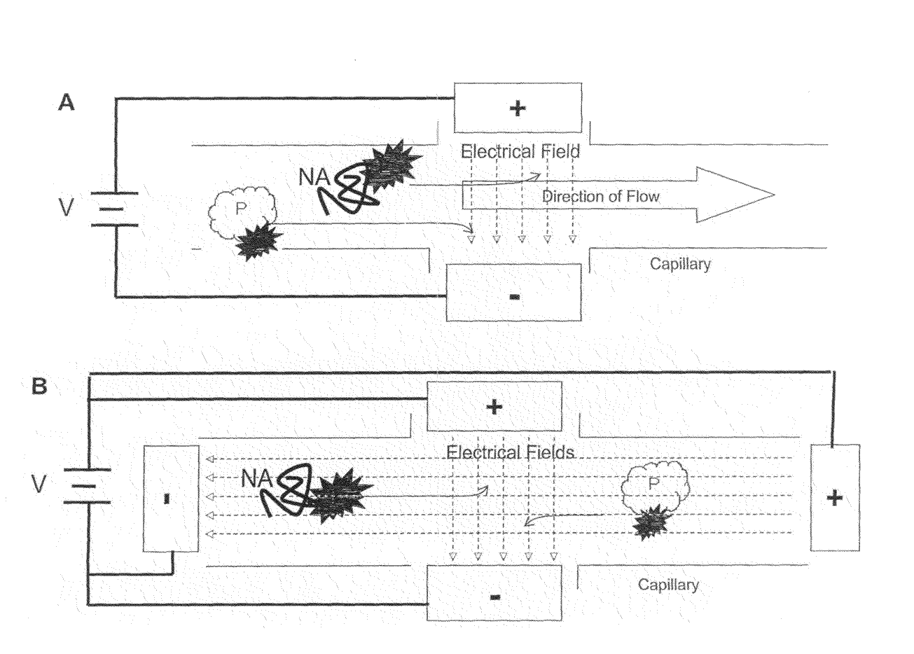

Protein and nucleic acid discrimination by mobility in one dimension is demonstrated in Example 1. One skilled in the art will recognize that molecular mobility can be further influenced by applying forces in more than one dimension. For example, applying an electrical field perpendicular to the axis of the capillary can cause molecules to move sideways within the capillary. The perpendicular electrical field can be applied continuously or intermittently and its polarity can be constant or reversed. The perpendicular field can be combined with any other motive force, such as electrophoresis, pumping and gravitational force, which is applied parallel to the capillary axis. One skilled in the art will recognize that movement from the center of the capillary to the side, or vice versa, will change the influence of the electroosmotic forces, which increase with proximity to the capillary wall, and ultimately the rate of molecular movement through the capillary. To the extent that the protein and nucleic acid are differentially affected by the additional force, their separation will be enhanced. FIG. 7 shows a diagram of an electric field applied perpendicular to the capillary axis while molecules are being moved along the capillary by pumping (7A) or electrophoresed (7B). In this example, the detector(s) must be downstream but near the location where the perpendicular force is applied. The detectors could also be located within the position of the perpendicular force. Alternatively, two detectors (and the interrogation volumes) could be stacked on the capillary, that is, oriented in a cross-sectional plane. In this case, the protein and nucleic acid may be co-detected as one species passes through one detector's interrogation volume, and the other species passes through the other detector's interrogation volume.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Such references shall include:
1. Alba, F. J., A. Bermudez, et al. (2001-2). “Green-light transilluminator for the detection without photodamage of proteins and DNA labeled with different fluorescent dyes.” Electrophoresis 22(3): 399-403.
2. Ambrose, W. P., P. M. Goodwin, et al. (1999). “Single molecule fluorescence spectroscopy at ambient temperature.” Chem Rev 99(10): 2929-56.
3. Brodsky, A. S. and P. A. Silver (2002). “A microbead-based system for identifying and characterizing RNA-protein interactions by flow cytometry.” Mol Cell Proteomics 1(12): 922-9.
4. Chiu, K. P., K. A. Duca, et al. (1996). “A novel in situ double-labeling method for simultaneous detection of mRNA and expressed protein or two different mRNAs.” J Neurosci Methods 66(2): 69-79.
5. Crissman, H. A., Z. Darzynkiewicz, et al. (1990). “Simultaneous fluorescent labeling of DNA, RNA, and protein.” Methods Cell Biol 33: 305-14.
6. Dunbar, S. A., J. W. Jacobson, et al. (2003). “Quantitative, multiplexed detection of bacterial pathogens: DNA and protein applications of the Luminex LabMAP™ system.” Journal of Microbiological Methods 53(2): 245-252.
7. Faretta, M., D. Bergamaschi, et al. (1998). “Characterization of cyclin B1 expression in human cancer cell lines by a new three-parameter BrdUrd/cyclin B1/DNA analysis.” Cytometry 31(1): 53-9.
8. Fischer, T. C., Q. T. Dinh, et al. (2002). “Simultaneous detection of receptor mRNA and ligand protein in human skin tissues.” J Cutan Pathol 29(2): 65-71.
9. Hasper, H. J., R. M. Weghorst, et al. (2000). “A new four-color flow cytometric assay to detect apoptosis in lymphocyte subsets of cultured peripheral blood cells.” Cytometry 40(2): 167-71.
10. Haugland, R. (2002). Molecular Probes Handbook of Fluorescent Probes and Research Chemicals, Molecular Probes Inc.
11. Holm, M., M. Thomsen, et al. (1998). “Optimization of a flow cytometric method for the simultaneous measurement of cell surface antigen, DNA content, and in vitro BrdUrd incorporation into normal and malignant hematopoietic cells.” Cytometry 32(1): 28-36.
12. Jing, D., J. Agnew, et al. (2003). “A sensitive two-color electrophoretic mobility shift assay for detecting both nucleic acids and protein in gels.” Proteomics: 1172-80.
13. Kellar, K. L. and M. A. Iannone (2002). “Multiplexed microsphere-based flow cytometric assays.” Exp Hematol 30(11): 1227-37.
14. Keller, R. A., W. P. Ambrose, et al. (2002). “Analytical applications of single-molecule detection.” Anal Chem 74(11): 316A-324A.
15. LeCaptain, D. J., M. A. Michel, et al. (2001). “Characterization of DNA-protein complexes by capillary electrophoresis-single molecule fluorescence correlation spectroscopy.” Analyst 126(8): 1279-84.
16. Ma, Y., M. R. Shortreed, et al. (2000). “High-throughput single-molecule spectroscopy in free solution.” Anal Chem 72(19): 4640-5.
17. Matray, T., V. Hernandex, et al., (2004). ELECTROPHORETIC TAG REAGENTS COMPRISING FLUORESCENT COMPOUNDS. US, Aclara Biosciences, Inc. Mountain View, Calif.
18. Nagaso, H., T. Murata, et al. (2001). “Simultaneous detection of RNA and protein by in situ hybridization and immunological staining.” J Histochem Cytochem 49(9): 1177-82.
19. Niculescu, A. B., 3rd, X. Chen, et al. (1998). “Effects of p21 (Cip1/Waf1) at both the G1/S and the G2/M cell cycle transitions: pRb is a critical determinant in blocking DNA replication and in preventing endoreduplication.” Mol Cell Biol 18(1): 629-43.
20. Perrin, A., D. Duracher, et al. (2003). “A combined oligonucleotide and protein microarray for the codetection of nucleic acids and antibodies associated with human immunodeficiency virus, hepatitis B virus, and hepatitis C virus infections.” Anal Biochem 322(2): 148-55.
21. Schwille, P. and E. Haustein (2002). Fluorescence Correlation Spectroscopy: An introduction to its concepts and applications. Single molecule techniques. P. Schwille. Bethesda, Md. http://www.biophysics.org/biophys/society/btol, Biophysics Textbook On-Line, Biophysical Society.
22. Waters, W. R., K. R. Harkins, et al. (2002). “Five-color flow cytometric analysis of swine lymphocytes for detection of proliferation, apoptosis, viability, and phenotype.” Cytometry 48(3): 146-52.
23. Wrotnowski, C. (2002). “Next-generation single molecule detection.” Genetic Engineering News 22: 28-27.

1. A method for co-detecting a polynucleotide molecule and a polypeptide molecule contained within one sample comprising:
(a) moving said polynucleotide molecule and said polypeptide molecule through at least one interrogation volume; and
(b) measuring at least one electromagnetic characteristic of said polynucleotide molecule and measuring at least one electromagnetic characteristic of said polypeptide molecule,
wherein said polynucleotide molecule and said polypeptide molecule are co-detected.

2. A method according to claim 1, wherein the polynucleotide molecule and the polypeptide molecule are discriminated.

3. A method according to claim 1, wherein the electromagnetic characteristic of the polynucleotide molecule and polypeptide molecule are measured simultaneously or sequentially.

4. A method according to claim 1, wherein a plurality of the same polynucleotide molecule and a plurality of the same polypeptide molecule are co-detected.

5. A method according to claim 1, wherein a plurality of different polynucleotide molecules and a plurality of different polypeptide molecules are co-detected.

6. A method according to claim 1, wherein a plurality of the same or different polynucleotide molecules and a plurality of the same or different polypeptide molecules are co-detected.

7. A method according to claim 1, wherein the polynucleotide is selected from the group consisting of a single-stranded DNA, a double-stranded DNA, an oligonucleotide, an RNA, a dendrimer, a nucleic acid hybrid and any combination thereof.

8. A method according to claim 1, wherein the polypeptide is selected from the group consisting of an oligopeptide and a protein.

9. A method according to claim 1, wherein the polypeptide electromagnetic characteristic and the polynucleotide electromagnetic characteristic are measured within at least one interrogation volume, said interrogation volume being in electromagnetic communication with at least one detector and at least one excitation source.

10. A method according to claim 9, wherein the interrogation volume is fluidly connected to at least one other interrogation volume.

11. A method according to claim 9, wherein the sample comprises a plurality of the same or different polynucleotide molecules and a plurality of the same or different polypeptide molecules, and wherein act (b) further comprises:
measuring at least one of a first or a second electromagnetic characteristic of at least one polynucleotide molecule as the at least one polynucleotide molecule interacts with an excitation source within a first interrogation volume;
measuring at least one of a first or a second electromagnetic characteristic of the at least one polynucleotide molecule as the at least one polynucleotide molecule interacts with an excitation source within a second interrogation volume;
comparing the measured electromagnetic characteristics measured within the first and the second interrogation volumes of the at least one polynucleotide;
measuring at least one of a first or a second electromagnetic characteristic of the at least one polypeptide molecule as the at least one polypeptide molecule interacts with an excitation source within the first, the second or a third interrogation volume;
measuring at least one of a first or a second electromagnetic characteristic of the at least one polypeptide molecule as the at least one polypeptide molecule interacts with an excitation source within the first, the second or a fourth interrogation volume; and
comparing the measured electromagnetic characteristics measured within the first, the second, the third and the fourth interrogation volumes of the at least one polypeptide.

12. A method according to claim 11, wherein the act of comparing comprises distinguishing by statistical analysis the measured electromagnetic characteristics of the at least one polypeptide molecule and the at least one polypeptide molecule from background electromagnetic characteristics.

13. A method according to claim 12, wherein the act of comparing comprises cross-correlating the measured electromagnetic emissions determined from the at least one polypeptide molecule and the at least one polypeptide molecule to determine the velocity of the molecules.

14. A method according to claim 12, wherein the act of comparing comprises cross-correlating the measured electromagnetic emissions to determine the velocity of the molecules.

15. A method according to claim 1, wherein act (a) comprises subjecting the sample to a motive force selected from the group consisting of electro-kinetic, pressure, vacuum, surface tension, gravity, centrifugal, and any combination thereof.

16. A method according to claim 15, wherein the act of moving the molecules between a first interrogation volume and a second interrogation volume further comprises subjecting the molecules to a separation method selected from the group consisting of capillary gel electrophoresis, micellar electro-kinetic chromatography, isotachophoresis, and any combination thereof.

17. A method according to claim 1, wherein at least one of the measurable characteristics of the polynucleotide molecule and at least one of the measurable characteristics of the polypeptide molecule is produced by one of an intrinsic parameter of the molecule and an extrinsic parameter of the molecule.

18. A method according to claim 17, further comprising marking the polynucleotide molecule and the polypeptide molecule with at least one label to provide the extrinsic parameter.

19. A method according to claim 18, wherein the polynucleotide molecule and polypeptide molecule are labeled prior to performing act (a).

20. A method according to claim 19, wherein at least one polynucleotide and at least one polypeptide are labeled in separate reactions and are combined to create the sample prior to performing act (a).

21-91. (canceled)
